(19) United States
(12) Patent Application Publication
(10) Pub. No.: US 2023/0343467 A1
(43) Pub. Date: Oct. 26, 2023

(54) SYSTEMS AND METHODS FOR IDENTIFICATION OF CELL LINES, BIOMARKERS, AND PATIENTS FOR DRUG RESPONSE PREDICTION

(71) Applicants: THERAINDX LIFESCIENCES PVT LTD, Karnataka Bangalore, (IN); ONCODYNAMIX LIFESCIENCES PVT LTD, Karnataka Bangalore (IN)

(72) Inventors: Ramesh JAYARAMAN, Karnataka Bangalore (IN); Sundaresh BABU, Karnataka Bangalore (IN); Priyanka BHARGAV, Karnataka Bangalore (IN); Mahesh NANJUNDAPPA, Karnataka Bangalore (IN)

(21) Appl. No.: 18/044,352

(22) PCT Filed: Sep. 6, 2021
(86) PCT No.: PCT/IN2021/050863

§ 371 Date: Mar. 7, 2023

(30) Foreign Application Priority Data
Sep. 7, 2020 (IN) 202041038569

(51) Int. Cl. G16H 50/70; G16B 5/00; G16H 20/10; G16H 10/20; G16H 70/40; G16H 50/30; G16H 50/20

(52) U.S. Cl. CPC G16H 50/70; G16B 5/00; G16H 20/10; G16H 10/20; G16H 70/40; G16H 50/30; G16H 50/20

(57) ABSTRACT

Methods for selection and identification of cancer cell lines, patients for drug screening and biomarkers, and prediction of clinical response in cancer patients are disclosed herein. The present invention relates to drug discovery and development and personalized medicine, and more particularly to systems and methods (300) for anti-cancer drug discovery and development for identification, selection and validation of a subset of commercially available/new human cancer cell lines for screening inhibitors or drugs, based on functional status of the relevant pharmacological target and its associated regulators/effectors in human cancer cell lines. It further discloses methods and systems (100) to predict clinical response of patients to drugs based on multi-omics, drug master networks and pathways for precision medicine in cancer.

### CROSS REFERENCE TO RELATED APPLICATIONS

This application is based on and derives the benefit of Indian Provisional Application 202041038569 filed on the 7 of Sep. 2020, the contents of which are incorporated herein by reference in their entirety.

### TECHNICAL FIELD

Embodiments herein relate to drug discovery and precision medicine in cancer, and more particularly to drug efficacy and clinical response prediction. It relates to systems and methods for identification and selection of cell lines and biomarkers (efficacy biomarkers and response assessment biomarkers), drug positioning (indication selection and patient selection) in drug discovery and development. It further relates to systems and methods for predicting drug response in selected cell lines or patient populations.

### BACKGROUND

In the early stages of cancer drug discovery programs, especially at the lead identification stage for targeted therapies, selection of appropriate cell lines, for e.g.: cancer cell lines of a specific cancer type, is crucial in understanding the mechanism of action of lead molecules. It is essential to demonstrate the proof of concept at a cellular level before progressing molecules to in vivo pharmacology studies. The proof of concept at the cellular level is the demonstration of inhibition of the pharmacological target which consequently results in the inhibition of the biochemical processes leading to a given pathology (for example, inhibition of cell growth and proliferation or angiogenesis, in cancer).

In cancer drug discovery, most cancer programs normally choose cancer cell lines in which the pharmacological target is over expressed or aberrantly active or in an empirical way, to screen compounds or drugs on standard set of cell lines. Such an approach mostly does not consider the pharmacological target, upstream and downstream regulators of the pharmacological target or parallel pathways. The expression and functions of the regulators influence the activation and activity of the pharmacological target which consequently affects the efficacy of the inhibitor of the target (lack of inhibition or resistance) in a significant manner. Therefore, the cancer cell lines to be selected for screening of inhibitors of the target should be based not only on the over-expression of the target but also the status of all the identified regulators of the pharmacological target.

Further, predicting response of cell lines or patient population to a particular drug candidate also plays a crucial role in identifying suitable candidates for preclinical or clinical studies. Early stage prediction based on genomics and multi-omics data, drug-target interaction, and pharmacokinetic properties provides a preliminary reference point in drug discovery.

Various technologies capable of selecting cell lines and in vitro screening have been developed based on cancer sub types. There are applications, which utilize the genomic diversity of the patient cohort for identification and selection of cell lines. In most of these approaches, however, cell lines are selected for screening compounds based on over-expression or aberrant expression of the relevant pharmacological targets. Further, applications capable of predicting patient response to treatment also exist. However, most of these approaches do not consider all factors and pathway features that can significantly impact drug efficacy. As a result, drug candidates or drugs may not always show efficacy in the selected cell lines. Even if drug candidate show response in smaller sub set of cell lines, it may fail to show response in larger cohort or in cohort with alterations in drug target or regulators (upstream, downstream, parallel pathways) of drug target. Therefore, there exists a need for a system that is capable of rational cell line selection based not only on aberrant or hyper-expressed pharmacological targets, but also based on the regulators and cell signaling network pathways that are linked to the target. Also, there exists a need for an integrated system capable of predicting drug response in selected cell lines or patient populations in drug discovery and development.

Clinical response to targeted anti-cancer therapy is highly variable. Various companion diagnostics for precision medicine in cancer use genomics-based approaches to identify clinically actionable mutations in tumor samples from cancer patients and recommend clinically approved targeted therapies to treat cancer patients. However, treatment of a patient with a drug based on clinically actionable mutations is not always correlated with clinical response. This is because the action of a drug is not always dependent on the aberrant expression of the pharmacological (drug) target, but also on the functional status of upstream regulators and downstream effectors that are linked to the drug target. Thus, the efficacy of a drug is based not only on the status of the target but also the components of the drug action pathway. There is an urgent need for pathway-based technologies to predict clinical response of patients to specific drugs based on their drug target-pathway status.

### OBJECTS

The principal object of the embodiments disclosed herein is to provide systems and methods for identification and selection of biomarkers, cancer cell lines, and relevant patient and/or indication for a drug candidate, drug or drug combination, in drug discovery and development.

Another object of the embodiments herein is to disclose systems and methods capable of integrating, in a mechanism based manner, the aberrantly expressed pharmacological target with its associated regulators and effectors and their functional status in human cancer cell lines, and selecting cancer cell lines for in vitro screening, particularly to demonstrate the effectiveness of the compound of interest, identify possible resistance loops that can build in, validate various hypothesis built during pathway analysis, identify indirect/novel mechanisms for compound sensitivity/resistance. The identified subset of cell lines is a representation of global cohort (all cell lines) in terms of alteration of all genes (part of query) and patterns of alterations of gene (combination of alterations) related to the pathway.

Another object of embodiments herein is to disclose systems and methods for selection of subset of human cancer cell lines for screening inhibitors (compounds) or drug of interest, based alteration of genes related to pharmacokinetics of inhibitors including drug efflux, import and metabolism in human cancer cell lines to understand the effect of genes influencing drug pharmacokinetics on intra cellular availability of compound of interest, in their altered state, in cancer condition.

Another object of embodiments herein is to disclose a method for constructing a ‘Master drug network’ around pharmacological target(s) with pathway components (enzymes and transcription factors), and identification of possible resistance/sensitive loops for a drug or compound of interest.

Another object of embodiments herein is to disclose a method to identify statistically significant multi-omics de-regulation patterns of biomarker(s) or a combination of biomarkers responsible for enhancing sensitivity or resistance (based on IC₅₀ values obtained for drugs in cell lines) of cancer cell lines to the compounds or drug of interest.

Yet another object of embodiments herein is to disclose a method for identification of suitable drug candidate, or drug or drug combination by integration of multi-omics data from cancer patients, pharmacokinetic data from patients, ex vivo (patient primary cell lines, organoids, tumors) and in vivo models of cancer (with patient tumors/cells), and to enable classification of individual patients, in terms of Drug Master Networks.

Another object of embodiments herein is to disclose a system capable of integration of multi-omics data from cancer patients, pharmacokinetic data from patients, ex vivo (patient primary cell lines, organoids, tumors) and in vivo models of cancer (with patient tumors/cells) to enable classifying individual patients, in terms of Drug Master Networks, for identification of suitable treatments with an appropriate drug or drug combinations.

An object of the embodiments herein is to disclose a system for predicting response of a cell line or a cancer patient to a particular drug or drug combination comprising a multilevel prediction model based on various factors such as artificial intelligence models, multi-omics data, standard biological rules, established guideline-based prediction, etc.

These and other objects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

The embodiments disclosed herein are illustrated in the accompanying drawings. The embodiments herein will be better understood from the following description with reference to the drawings, in which:

FIG. 1 depicts an example system 100 configured to predict patient response to a drug or a combination of drugs of interest, according to embodiments as disclosed herein;

FIG. 2 depicts an example system 200 configured for identification and selection of biomarkers, cell lines, and relevant patient and/or indication for a drug candidate, drug or a combination of drugs, according to embodiments as disclosed herein;

FIGS. 3A and 3B are schematic representations of method 300 for identification, selection and validation of Human Cancer Cell lines and Biomarkers, according to embodiments as disclosed herein;

FIG. 4 is pictorial representation of the method 300 for identification, selection and validation of Cancer Cell lines and biomarkers based on Pharmacological Target(s), patient stratification/selection in clinical trials, biomarkers to be monitored during treatment, according to embodiments as disclosed herein;

FIG. 5 shows an example diagram of drug master network built for CDK4/6 inhibitors for MCF7 breast cancer cell line;

FIG. 6 shows an example diagram of drug master network built for CDK4/6 inhibitors for HCC2157 breast cancer cell line;

FIG. 7 illustrates the alteration patterns in genes of interest and few representative cell lines selected for each pattern;

FIG. 8 is a flowchart representation of the method of selection of cell lines with alteration in drug transporters;

FIG. 9 is a graphical representation of predicted response curves of cell line population with amplified ABCB4 gene as compared to the cell line population with wildtype ABCB4 for drug vincristine;

FIG. 10 is a graphical representation of predicted response curves of cell line population with amplified ABCB1 gene as compared to the cell line population with wildtype ABCB1 for drug vincristine;

FIG. 11 is a graphical representation of response curves of the patient populations with mutant runx1 gene and wildtype runx1 gene as predicted by the response predictor for drug combination 7+3 (standard induction chemotherapy—AML), according to embodiments herein;

FIG. 12 is a graphical representation of response curves of the patient population with amplified asxl1 gene and wildtype asxl1 gene as predicted by the response predictor for drug combination 7+3 (standard induction chemotherapy—AML), according to embodiments herein;

FIG. 13 is a graphical representation of response curves of patient population with amplified mutant tp53 gene and wildtype tp53 gene as predicted by the response predictor, for drug combination 7+3 (standard induction chemotherapy—AML) according to embodiments herein;

FIG. 14 is a graphical representation of response curves of the patient population with amplified mutant npm1 gene and wildtype npm1 gene as predicted by the response predictor for drug combination 7+3 (standard induction chemotherapy—AML), according to embodiments herein;

FIG. 15 is clinical data interpretation protocol used for classifying patients into clinical responders and non-responders for validation of response prediction, in high-grade serous ovarian cancer dataset (HGS-OvCa);

FIGS. 16 and 17 are example diagrams of a drug master network for the platinum-based drug, Cisplatin, created by the system according to embodiments herein;

FIG. 18 is an example diagram depicting the key pathways as identified by the system, according to embodiments herein, with the drug master network created for platinum-based drug Cisplatin;

FIG. 19 is an example diagram depicting other relevant pathways and biomarkers as identified by the system, according to embodiments herein, by the drug master network created for platinum-based drug Cisplatin;

FIG. 20 depicts the weight ages given for each parameter in order to predict response of patients to drug, Cisplatin;

FIG. 21 is a schematic representation of the workflow for the multi-omics based stratification process;

FIG. 22 is a graphical representation of predicted response curves of the cell line population with amplified CCND1 gene as compared to the cell line population with wildtype CCND1 gene for drug Palbociclib;

FIG. 23 is a graphical representation of predicted response curves of the cell line population with amplified CDK4 gene as compared to the cell line population with wild type CDK4 gene for drug Palbociclib;

FIG. 24 is a graphical representation of predicted response curves of the cell line population with deleted CDKN2A gene as compared to the cell line population wildtype CDKN2A gene for drug Palbociclib;

FIG. 25 is a graphical representation of the predicted response curves of the cell line population with deleted RB1 gene as compared to the cell line population wildtype RB1 gene for drug Palbociclib;

FIG. 26 is a graphical representation of the predicted response curves of the cell line population with amplified CCNE1 gene as compared to the wildtype CCNE1 gene cell line population for drug Palbociclib;

FIG. 27 is a graphical representation of the predicted response curves of the cell line population with a loss of function mutation for the gene FAT1 gene as compared to the wildtype FAT1 gene cell line population for drug Palbociclib;

FIG. 28 is a graphical representation of response curves of the cell line population showing the effect of amplification of CDKN1A gene in response to the drug Palbociclib, predicted by the system according to embodiments herein;

FIG. 29 is a graphical representation of response curves of the cell line population showing the effect of deletion of CDKN1A gene in response to the drug Palbociclib, predicted by the system according to embodiments herein;

FIG. 30 is a schematic representation of an example workflow depicting the integrated approach of response prediction, according to embodiments herein;

FIG. 31 is a flowchart depicting a patient clinical data interpretation of a retrospective dataset—BEAT AML, where patients are classified into responders and non-responders based on clinical data. This was used to compare and validate response prediction by a system according to embodiments herein;

FIG. 32 is an example drug master network of 7+3 standard induction chemotherapy drug system, by a system according to embodiments herein; and

FIG. 33 is a representation which depicts assigning response scores for response prediction based on biological rules in 7+3 standard induction chemotherapy drug system, according to embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein disclose systems and methods for drug discovery and development in cancer, and for precision medicine in cancer. While embodiments herein are illustrated with reference to the field of cancer and cancer therapy, it is understood that the embodiments herein are helpful and contemplated for use in drug discovery and development and precision medicine in any field of medicine. Embodiments herein include systems and methods useful in identification, selection and prediction application in the various known phases of drug discovery and development. Further, embodiments herein may also be used to facilitate prediction of patient responses to drug candidates still under trial and/or in drug discovery stage, new and/or approved drugs and drug combinations, and therefore is contemplated for use by users such as health experts, clinicians, researchers, etc. in structuring and planning personalized or precision medication and therapeutic approaches. The system, according to embodiments herein, takes a layered integrated approach in structuring and analyzing diverse data to provide a user-based output.

The technical and scientific terms used herein have the same meaning as generally understood to a person skilled in the art. The term “multi-omics”, as used herein, refers to its generally accepted meaning in its broadest sense. It refers to an approach of biomolecular analysis at multiple levels such as genomics, epigenomics, transcriptomics, proteomics, metabolomics, biological networks and system models. Multi-omics data, as used to herein, broadly includes any data generated at such multi-omic level. Examples of the types of multi-omics data includes, but is not limited to, high through put data relating to RNA-Seq, DNA-Seq, microRNA, single-nucleotide variant (SNV), copy number variation (CNV), mRNA deregulation, methylation, fusion protein, biopsy, BMA, peripheral blood work, DNA methylation, reverse phase protein array (RPPA) , whole genome sequencing, genetic mutation, and genomic variations data (somatic and germline mutation), Clinical traits, gene expression, miRNA expression, copy number, and sequencing data. Such multi-omics data includes data generated inhouse and that available at various databases including, but not limited to, The Cancer Genome Atlas (TCGA), Clinical Proteomic Tumor Analysis Consortium (CPTAC), International Cancer Genomics Consortium (ICGC), Cancer Cell Line Encyclopedia (CCLE), Molecular Taxonomy of Breast Cancer International Consortium (METABRIC), TARGET, American Society of Clinical Oncology (ASCO), National Cancer Institute (NCI), cBioPortal and Omics Discovery Index. The term “genomics”, as used herein, refers to approaches at genome levels such as functional genomics, structural genomics, metagenomics, epigenetic, etc. Genomic data as used herein refers to data generated at such genome levels.

The term “indication”, as used herein, refers to any medical condition, disease or disorder. It refers to a condition usually known to be abnormal or have deleterious effects on a subject. In an embodiment, the indication is cancer. Examples of cancer includes, but is not limited to, brain tumor, colorectal cancer, lung cancer, mesothelioma, soft tissue sarcoma, pancreatic cancer, neuroendocrine tumor, glioblastoma, neuroblastoma, melanoma, skin cancer, colon cancer, rectal cancer, prostate cancer, head tumor, a neck tumor, breast cancer, a gynecological tumor, liver cancer, kidney cancer, lung cancer, bile duct cancer, cervical cancer, endometrial cancer, esophageal cancer, stomach cancer, medullary thyroid cancer, ovarian cancer , Glioma, lymphoma, leukemia, myeloma, acute lymphoblastic leukemia, acute myeloid leukemia, chronic lymphocytic leukemia, chronic myelogenous leukemia, Hodgkin lymphoma, non-Hodgkin lymphoma, urinary bladder cancer, etc.

Algorithm is defined as any set of instructions or methods that can be used iteratively or repeatedly to generate outcomes or results which are quantified and meaningful for analysis. The algorithm may be defined in terms of a set of instructions written in computer compatible language or code that reflects the rules and mathematical framework defined by the logic in the algorithm. Machine learning and Artificial Intelligence refers collectively to the set of methods, algorithms, modules and code written and executed with a specialized function or purpose. Machine Learning and Artificial Intelligence is reflected in the algorithm or method's utility in developing insights and analytical data points that are not intuitive or apparent in the information provided as the input.

The term “subject” or “patient”, used interchangeably herein refers to any human or other organism. It refers to human subjects having or suspected of having a medical condition. It also refers to any individual whose health, or pathological, clinical or other medical data is a subject of clinical or research interest, or research. It further includes cell lines used, for example in various stages of drug discovery and developments, to understand biochemical processes and/or drug response in humans. In an embodiment, the subject or patient is a cancer patient. The term “responders” and “non-responders” refers to its generally known meaning. It is used to identify a subject based on a subject's responsiveness or response to a particular drug, drug candidate, drug combination, etc.

The term “pathway”, as used herein broadly refers to biological pathways related to the human body, especially at a cellular or molecular level. It refers to metabolic pathways, signaling pathways, defense (Immunological) pathways, and the like, including parallel pathways, pathway cross talks, etc. It also refers to any pathway which may be affected or altered by a medical condition, foreign matter, therapeutic interventions, drugs, infections, etc., including regulated and deregulated pathways, or differentially regulated pathways. Example of pathways include mitogen activated protein kinase (MAPK) cell signaling pathway, Hippo pathway, RAF-ERK signaling pathway, etc.

The term “differential”, as used herein broadly refers to a change from normal or generally known condition or form. It may be relative or absolute. A pathway feature refers to a feature in such pathway, especially capable of being affected by alterations at multi-omic level, for e.g.: genetic alterations, change in mRNA expression, copy number variation, mutation, methylation, fusion, proteomic changes, etc. In an embodiment, pathway refers to any biological pathway related to or associated with a pharmacological target for cancer. The term “target” as used herein refers to any pharmacological target. It refers to any biomolecule which directly or indirectly is capable of being affected to bring about a pathological change. Examples of such targets include, but are not limited to, Cyclin Dependent Kinase 4/6 (CDK4/6), Receptor Tyrosine Kinases, Transcription factor E2F1, etc.

The term “mutation” refers to its broadest generally known meaning in the art. It refers to genetic or genomic alterations which may or may not result in a deviation in functionality of gene product. Based on functionality, mutation may be broadly classified as GOF (Gain of Function), LOF (Loss of function), COF (Conservation of function) and SOF (Switch of function). Examples of such include, but are not limited to, F595L, A728V, G465A, etc.

The term, “reference”, as used herein, refers to any value or data which may be used in comparative analysis. It may include in vitro data, drug response data, drug sensitivity data, patient genomics signatures, protein expression patterns, pre-clinical data, drug efficacy data, etc. It may include data or datasets which may be obtained from a storage source (such as a database or a repository), or healthy subjects. Examples of databases include databases by institutes such as Cancerrx (database of genomics of drug sensitivity), The Cancer Genome Atlas (TCGA), Clinical Proteomic Tumor Analysis Consortium (CPTAC), International Cancer Genomics Consortium (ICGC), Cancer Cell Line Encyclopedia (CCLE), Molecular Taxonomy of Breast Cancer International Consortium (METABRIC), TARGET and Omics Discovery Index, American Society of Clinical Oncology (ASCO), National Cancer Institute (NCI), etc. In an embodiment, it is obtained from an in-house database. The reference data may also be used in training a system or module to develop a pattern for analyzing samples. In certain embodiments, biological rules are used as reference. Biological rules, as used herein, include hypothesized rules based on the biology of target, drug characteristics, pathway and target regulation, cellular efflux and influx pump information, pathological data, target sensitivity and resistance data, pathway effectors and regulators data, and drug-target interaction. In certain embodiments, standard guidelines may be used as reference. Standard guidelines, as used herein, includes guidelines by certified and/or known authorities such as US Federal Drug Administration (USFDA), National Comprehensive Cancer Network (NCCN) Guidelines, European LeukemiaNet (ELN) recommendations, etc.

The term “samples”, as used herein, refers to any biological specimen including a biomolecule. It includes samples derived from biological fluids, cells, tissues, organs or organisms. It refers to any sample which may be used to assess health of a subject, a particular medical condition, disease state, response to therapy, risk of developing a disease, etc. In an embodiment, it refers to tumor cell samples such as biopsy, bone marrow samples, peripheral blood, liquid biopsy tissue, Circulating tumor cells (CTCs) etc.

The term “biomarker”, as used herein, refers to an indicator for example a biomolecule, such as a nucleic acid, enzyme, protein or protein fragment, peptide in a subject; or state of a biomolecule, such as gene expression pattern, nucleic acids methylation, enzyme phosphorylation, etc., wherein the quantity, concentration, status, or activity of the biomarker in the subject provides information of diagnostic and prognostic value, for example about the subject's response to a drug candidate, drug or drug combination; and/or whether the subject has, or is at risk of developing, a medical condition or a disease state; etc. It includes its meaning as per National Cancer Institute, as “a biological molecule found in blood, other body fluids, or tissues that is a sign of a normal or abnormal process, or of a condition or disease,” such as cancer. In an embodiment, biomarkers include tumor biomarkers.

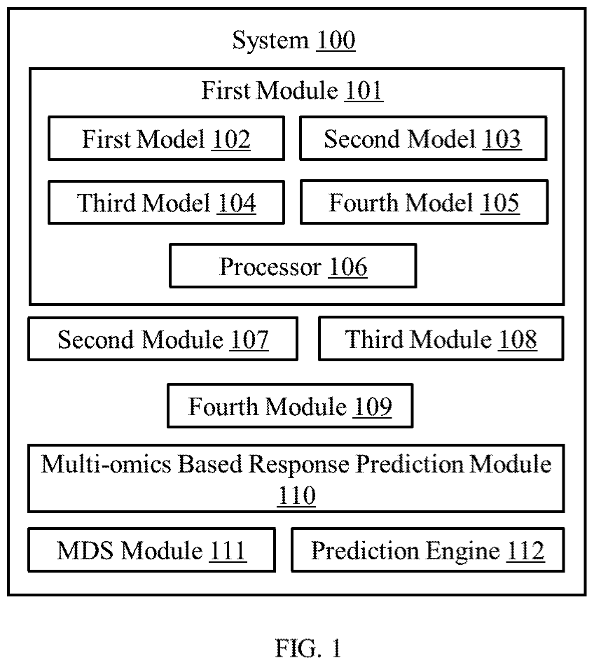

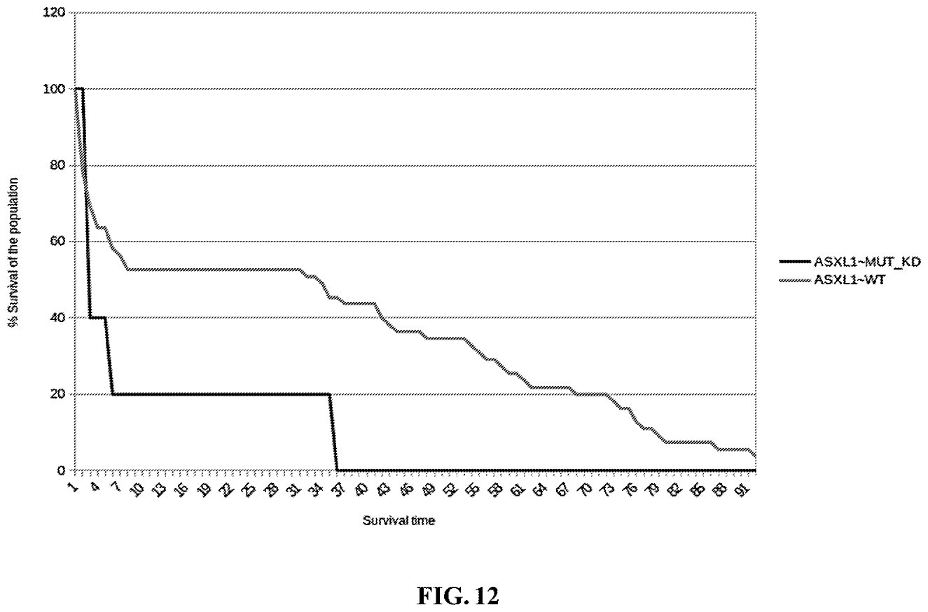

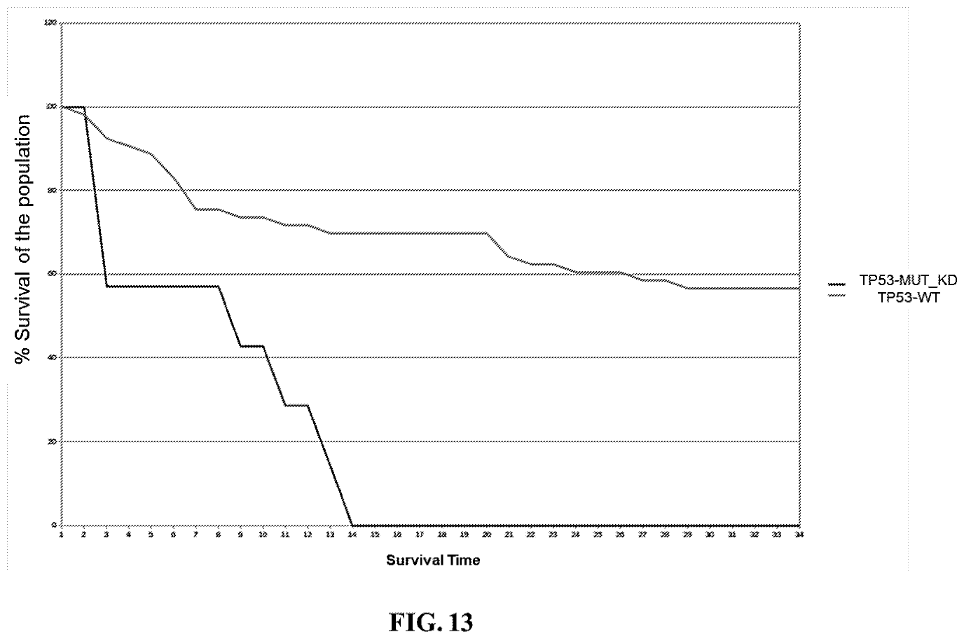

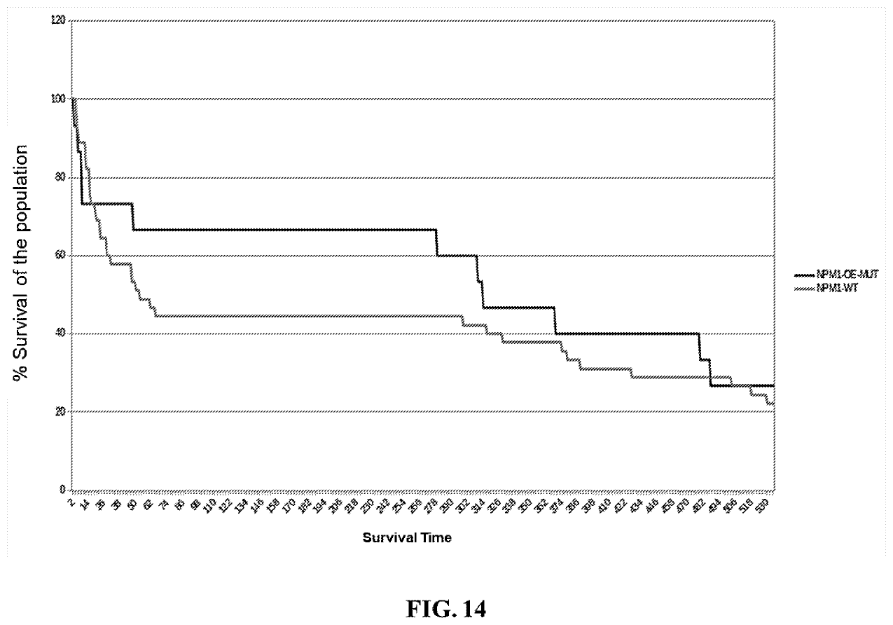

Referring now to the drawings, and more particularly to FIGS. 1 through 33, where similar reference characters denote corresponding features consistently throughout the figures, there are shown embodiments.

FIG. 1 depicts an example system 100 configured to predict a subject's response to a drug candidate, drug or a combination of drugs, particularly in cancer, according to embodiments as disclosed herein. The system 100 can be referred to as an integrated system for response prediction and precision medication as the prediction is performed at three or four levels to achieve a final prediction on patient response to a drug or drug combination or drug candidate. Embodiments herein are contemplated to provide support in making treatment decision or precision medication. It may further be useful in patient's prognosis to a particular drug/drug combination or drug candidate. Accordingly, embodiments herein include a system and method for prognosis. As depicted in FIG. 1, the system 100 comprises a plurality of modules comprising a first module 101, a second module 107, a third module 108, and a fourth module 109, for predicting the patient response. In an embodiment, the system 100 performs a first level prediction using the first module 101. In an embodiment, the system 100 performs a second level prediction using the second module 107. In an embodiment, the system 100 performs a third level prediction using the third module 108. In an embodiment, the system 100 utilizes the fourth module 109 as an optional fourth level prediction module.

The first module 101 uses multi-omics data for determining patient response at a first level. The first module 101 includes at least one of a first model 102, a second model 103, a third model 104, a fourth model 105, and a processor 106. The first model 102 can determine a first factor comprising at least one pathway feature. The second model 103 can determine a second factor defining a similarity between at least one gene alteration feature. The third model 104 can determine a third factor comprising a plurality of decision trees. The fourth model 105 can determine a fourth factor defining a similarity determined using a nearest neighbor technique. The processor 106 can predict the response of the subject based on at least one of the first factor, the second factor, the third factor, and the fourth factor. The processor 106 can assign weights to each of the first factor, the second factor, the third factor, and the fourth factor, which can influence an outcome of the prediction.

The multi-omics gene data, as described previously, refers to any patient data at genomic, transcriptomics, proteomic and/or metabolomic level, including, but not limited to mutation data, copy number variation data, m-RNA deregulation data, methylation data, fusion protein data, and other non-genomic data such as Microsatellite instability, Tumor mutational burden, blood parameters, etc. Multi-omics data may be obtained by standard reference databases and libraries and/or in house data and/or at least one module disclosed herein. Genomic mutation data is analyzed using a MDS module 111 for functionality prediction.

The multi-omics-based response prediction module 110 can predict the response to a drug or drug of interest based on multi-omics of a patient. An in-house patient database, having response and multi-omics data, may be used in order to train the multi-omics-based response prediction module 110 for a particular drug or drug of interest, which may then be used to identify the genes and pathways that are differentially regulated in responders and non-responders. Differentially regulated gene and pathway patterns are further mapped with reference datasets to predict response of a patient to a particular drug or drug of interest. For example, in an embodiment, for predicting response of a patient to the standard Cytarabine and Daunorubicin, with 7+3 (i.e., 7 days of Cytarabine and 3 days of Daunorubicin) treatment regimen, the multi-omics-based response prediction module 110 is trained with standard response data of AML patients; for example, a response of a patient can be predicted based on multi-omics match, using the data from over 400 patients. In an embodiment, the first module 101 integrates results of prediction obtained from the first model 102, the second model 103, the third model 108, and the fourth model 105, by assigning weightage to each of the first model 102, the second model 103, the third model 108, and the fourth model 105, based on relevance and accuracy of each of the first model 102, the second model 103, the third model 108, and the fourth model 105, to predict an outcome of response of a patient to a drug/combination of drugs.

The MDS module 111 can predict the functionality of a mutation, based on genomics of a patient. Mutation functionality prediction may be used in precision medication or targeted therapeutics. Embodiments herein are capable of predicting functionality of mutation by construction activity projections of wild type and mutant protein model, and pattern recognition protocol.

The second module 107 utilizes standard guidelines for determining patient response at a third level. In an embodiment, the second module 107 can be referred to as Guideline based patient risk stratification module. The second module 107 is meant for stratifying patients into various risk zones based on predefined or established guidelines or observations relating to genomic alterations. An example of such guidelines include risk zone stratification of AML patients into low/intermediate/high risk zones based on genomic alterations i.e. patients with RUNX1_RUNX1T1, CBFB_MYH11 fusions and/or NPM1 mut FTL3 wild type/FLT3-itd (low allelic frequency) and/or biallelic mutated CEBPA in low risk zone, patients with NPM1 mut FTL3-itd (high allelic frequency) and/or NPM1 wild type FTL3 (low allelic frequency) and/or MLLT3_KMT2A fusion into intermediate risk zone, patients with DEK_NUP214, KMT2A rearranged, BCR_ABL1, GATA_MECOM fusion and/or 5q del and/or 7 del and/or −17p del and/or complex karyotype and/or monosomal karyotype and/or FLT3 itd (high allelic frequency) NPM1 wild type and/or RUNX1 mutant and/or TP53 mutant and/or ASXL1 mutant into high risk zone. The patient may be identified as of favorable risk, intermediate risk and adverse risk based on genomic characteristics. The pre-defined guidelines may vary based on the drug and/or indication. In an example, National Comprehensive Cancer Network (NCCN) guidelines and European LeukemiaNet (ELN) recommendations are used in stratifying AML patients in various risk zones based on patient genomics. Determining the level of risk of a patient is known to play a crucial role in deciding treatment approaches and patient prognosis. If guidelines are not present for any drug/combination, embodiments herein use the stratification module 107 for identifying statistically significant biomarkers for risk zone classification.

The third module 108 utilizes biological rules for determining patient response. In an embodiment, the third module 108 can be referred to as rule based response predictor module. Prediction of patient's response is based on biological rules that may be used to train the third module 108. The biological rules include, but is not limited to, drug chemistry, drug-target interaction biology, drug target alterations, effector and regulator functional status, drug influx and efflux data, target metabolic pathways and drug metabolism. Further, genomic and multi-omics alteration and differential pathway signatures are assigned weightages based on their importance in drug function to arrive at a final rule equation which is defined to predict a score for a particular drug response. For FTL3 inhibitor, multi-omics alterations responsible for drug sensitivity include FLT3-itd mutant/tkd mutant and KIT mutant. Multi-omics alterations responsible for drug resistance include FLT3LG over expression, CYP3A4 over expression, RAS/RAF mutant, PIK3CA mutant, PTEN mutant/loss of expression, IL3/GMCSF over expression, JAK mutant/over expression, SOCS mutant/loss of expression/CCND3 mutant/BIRC5/PIM1/PIM2 over expression. These deregulations are used as rules to determine whether or not a patient will respond to the drug based on patient specific alterations in these regulators. The third module 108, is capable of determining patient and/or cell line response to a drug candidate, drug or drug combination by analyzing multi-omics alterations in a patient and/or cell line using guidelines based on deregulations.

In an embodiment, the system 100 includes a prediction engine 112. The prediction engine 112 can combine the predictions of the first module 101, the second module 107, and the third module 108 based on priorities corresponding to the predictions performed by each of the first module 101, the second module 107, and the third module 108.

The fourth module 109 utilizes laboratory results for determining and/or validating patient response at a fourth level. Patient derived sample, for examples: tumor cells or tumor samples, are subjected to laboratory analysis. Laboratory analysis may include an array of tests generally known in the field, and may depend on the indication and drug, drug combination or drug candidate. The tests include, but are not limited to, cell line testing, cytotoxicity assays, biomarker assays, drug efflux and influx assays, animal model testing (Xenoraft and Hollow fibre models). Examples of tests include, but are not limited to, test for detection of cell viability/proliferation including tests based on cellular enzymes, based on DNA synthesis, Dye exclusion assays, Electric cell-substrate impedance sensing, Colony formation assays and real-time cell monitoring of cell proliferation and imaging; assays based on cellular enzymes and proteins such as MTT (3-(4,5-dimethylthiazol-2-yl)-2,5-diphenyltetrazolium bromide) assay, MTS (3-(4,5-dimethylthiazol-2-yl)-5-(3-carboxymethoxyphenyl)-2-(4-sulfophenyl)-2H-tetrazolium) assay, XTT ((2,3-bis (2-methoxy-4-nitro-5-sulfophenyl)-2H-tetrazolium-5-carboxanilide) assay, WST (Water Soluble Tetrazolium Salts) assay, SRB (sulforhodamine B) assay, Adenosine triphosphate assay, Lactate dehydrogenase leakage assay, Calcein, AM assay and Resazurin assay; assays based on DNA synthesis including [³H]-Thymidine incorporation assay, 5-Bromo-2′-deoxyuridine assay, 5-Ethynyl-2′-deoxyuridine assay and Mitotic index assay. Dye exclusion assays include Tryphan blue, Propidium iodide, 7-Aminoactinomycin D, STOX family dyes, SYTO family dyes and Ethidium homodimer; apoptosis based in vitro tests such as microscopic techniques (Light microscopy, Fluorescence microscopy and Confocal microscopy), Flow cytometry, DNA fragmentation and damage, Cytosolic calcium concentration, Detection of the expression of apoptosis related genes and proteins and Detection of caspases; angiogenesis based in vitro test such as Boyden chamber assay, Micro-carrier beads, Tube formation assay, Cell proliferation assay, Aortic ring assay and Matrix metalloproteinases activity; cell migration and invasion based in vitro test such as Wound healing assay, Boyden chamber assay, Capillary chamber cell migration assay, Cell Exclusion Zone Assay, Spheroid-based migration assay and Real-time cell monitoring of cell proliferation and imaging; oxidative stress based in vitro test include markers such as thiobarbituric acid reactive substances, 8-isoprostane, 8-hydroxy-2′-deoxyguanosine, Reactive oxygen species, Reactive nitrogen species, Concentration of hydrogen peroxide, Protein carbonylation, S-Nitrosylation, S-Glutathionylation and Glutathione; enzymes such as Nitric oxide synthases, catalase, Thioredoxin reductase and Glutathione-S transferases; Cellular senescence based in vitro test such as Telomere length, Telomeric repeat amplification protocol assay and Senescence-associated β-galactosidase activity; in vitro tests based on genomic alterations such as Metaphase chromosome analysis, Comparative genomic hybridization (CGH), Restriction landmark genomic scanning (RLGS) and Representational difference analysis (RDA); in vitro tests based on gene and protein expression include Real-time polymerase chain reaction, serial analysis of gene expression (SAGE), Cap analysis gene expression, Immunoassays, Immunoprecipitation and Western blotting; and in vitro tests based on Energy metabolism include Glucose uptake, Glycolytic enzymes, Oxygen consumption and Lactate assay.

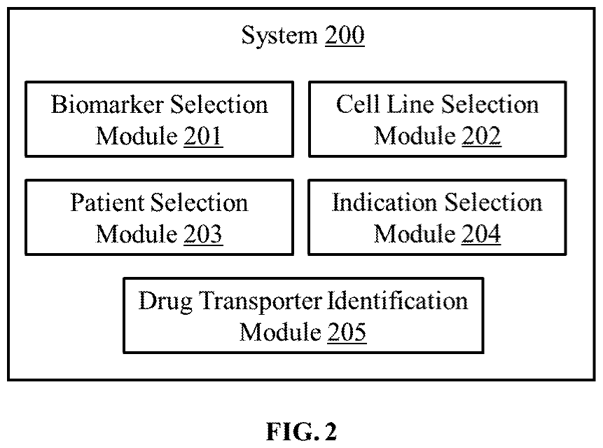

FIG. 2 depicts an example system 200 configured for identification and selection of biomarkers, cell lines, and patient and/or indication for a drug candidate, drug or a combination of drugs, according to embodiments as disclosed herein. As depicted in FIG. 2, the system 200 comprises a plurality of modules comprising a biomarker selection module 201, a cell line selection module 202, a patient selection module 203, an indication selection module 204, and a drug transporter identification module 205. In an embodiment, the biomarker selection module 201 is meant for identification of biomarkers based on pharmacological target(s), the cell line selection module 202 is meant for identification of appropriate cell line based on functional status of relevant pharmacological target and/or its associated regulators/effectors. The patient selection module 203 is meant for selection of patient population for a particular pathological target based on multi-omics data. The indication selection module 204 is meant for selection of indication relevant to a drug-target interaction using the patient population data. The drug transporter identification module 205 is meant for identifying co-relating factors that affect drug availability, and/or drug transportation in selected cell lines or cell line subsets based. Based on the target of the candidate drug molecule, drug master network is built with known regulators of the target—including upstream regulators, downstream effectors and parallel pathways. Converging biomarkers linking the drug target to phenotypic end points are selected as potential assessment biomarkers 201. Parallel pathways regulating the common downstream as the target of interest are identified as potential combination therapy targets for increasing the efficacy of the drug of interest. Cell lines are selected based on alterations of regulators in drug master network, such that the selected sub set has representatives with alterations of all the relevant genes in drug master network, making the selected sub set a good representative of global cohort 202. The selected cell lines are screened to obtain cell line specific IC₅₀ values—in vitro screening. The cell line specific IC₅₀ values and cell lines' multi-omics data is used by the stratification algorithm to identify statistically significant efficacy biomarkers determining drug sensitivity—favorable genomics/resistance—unfavorable genomics 206. The alteration frequency of these efficacy biomarkers is used to position the drug. Patients are selected with presence of favorable genomics and absence of unfavorable genomics 203. Indication is selected based on high frequency of patient population harboring favorable genomics and low frequency of patients harboring unfavorable genomics.

The system 200 allows identification, selection and validation of Cancer Cell lines and Biomarkers based on Pharmacological Target(s). Embodiments herein reduce lead time with significant cost saving at discovery stage by selection of appropriate cell lines or panel of cell lines, particularly cancer cell lines, for Lead Identification and Lead Optimization programs, identifies appropriate biomarkers for PK/PD and enables identification of appropriate patient population for clinical trials to optimize drug response. The system 200 provides a mechanistic approach for selection of a subset of commercially available/new human cancer cell lines for screening inhibitors (compounds) or drug of interest, based on functional status of relevant pharmacological target and its associated regulators/effectors in human cancer cell lines. The system 200 allows constructing a ‘Master drug network’ (also referred to herein as “Drug master network”) around a pharmacological target(s) with pathway components (enzymes and transcription factors). The system 200 allows identifying statistically significant multi-omics de-regulation patterns of biomarker(s) or a combination of biomarkers responsible for enhancing sensitivity or resistance (based on IC₅₀ values obtained for cell lines) of cancer cell lines to the compounds or drug of interest.

The system 200 allows usage of pathway analysis during early stage of drug discovery (before in vitro screening), with specific drug master network constructed around drug targets, with well-defined pathway regulations at upstream, downstream, parallel pathways and pathway cross talks. The pathways are different from biological pathways, as they are constructed specifically around Pharmacological (drug) target gene(s), with defined regulators upstream, downstream, by-pass and cross talks with respect to drug target. The drug master network will contain whole or a part of different biological pathways integrated together relevant to the pharmacological target(s). Integration of cancer cell line data to the master drug network is done at multi-omics level, to get a holistic view of alteration of drug pathway in each cell line. The pathway players are assigned weightages by examining—alteration frequency in cancer, impact on activation/activity of drug target gene(s) and its alteration impact of survival and expression of drug efflux transporters and drug metabolizing enzymes (patient population). In an embodiment herein, a ‘Master drug network’ is constructed around a pharmacological target, for cell line selection for in vitro screening, with pathway players including, but not limited to, upstream regulators, downstream effectors, genes regulating by pass mechanisms and pathway cross talks. These pathways players are either tagged as positive biomarkers or negative biomarkers based on their effect on activation (upstream) or activity (downstream) of pharmacological target. Cancer cell lines, with alterations in target, positive biomarkers, negative biomarkers and PK determinants are selected for screening by embodiments herein, which predicts drug response in selected cancer cell lines, later confirmed by in vitro screening. Embodiments herein use dual approach for identification and validation of biomarkers that drives/enhances sensitivity or resistance of a particular drug molecule to a particular cell line. The identified biomarkers are backed up with a scientific rationale. Embodiments herein use biomarkers (positive and negative) which drive or enhance drug sensitivity and resistance, integrated with patient population multi-omics data to select appropriate indications, based on high alteration frequency of drug sensitive loops, low alteration frequency of drug resistance loops, and considering co-occurrence and exclusivity patterns of biomarker alterations, for patient selection, and indication selection.

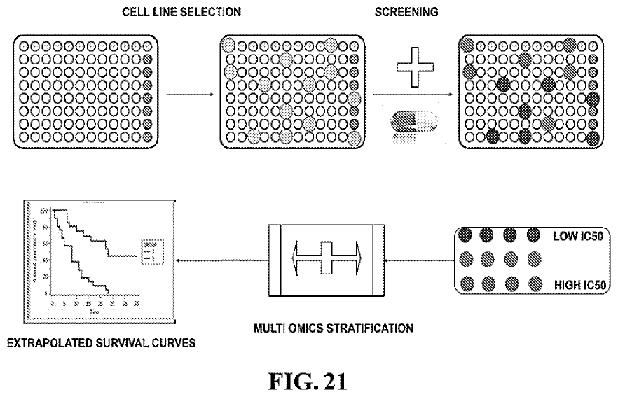

The patient selection module 203 and the indication selection module 204 allow identification of target population of patients and/or target indication, such as: cancer type, sub types, during early phase of drug discovery. Embodiments include identifying the target patient and/or indication based on the presence of favorable genomics in patients for a particular drug or drug candidate. The target patients and/or indication may be identified for a particular pathological target based on multi-omics data. Favorable and unfavorable genomics are identified post-screening from in vitro efficacy data, wherein the stratification algorithm as described in various embodiments herein identifies multi-omics such as: mutations, copy number variation (CNV), mRNA deregulations, and so on, which are responsible for enhanced drug sensitivity/drug resistance. The multi-omics based stratification process is schematically represented in FIG. 21. For example, in CDK4/CDK6 inhibitor, CDKN2A deep deletion and/or CDKN2B deep deletion and RB1 wild type status is identified as favorable genomics for drug Palbociclib. The panel of cell lines selected may include cell line subsets with alterations in genomics. In an embodiment, patient population data can be utilized for selecting indication types and/or subtypes possessing maximum number of patients with favorable genomics.

The drug transporter identification module 205 allows identification of co-relating genomic factors such as factors affecting drug transporters, transporter genes, or any alterations therein such as transporter gene amplification including copy number alterations (CAN), increased m-RNA expressions, etc. particularly associated with multidrug resistance (MDR) development. The drug transporter identification module 205 allows identifying drug transporter factors that impact the efficacy of the drug of interest using in vitro sensitivity data, during pre-clinical phase of drug development. Drug transporters are considered to be key determinants of drug accumulation within cells, whose activities are often directly related to therapeutic efficacy, drug toxicity, and drug-drug interactions. Drug transporters such as solute carrier (SLC) family and ATP-binding cassette (ABC) family are known to function as drug efflux pumps which contribute towards drug resistance and reduced bioavailability. The method, in various embodiments herein use in vitro sensitivity data in identification of MDR transporter genes during pre-clinical phase of drug development. In an embodiment, in vitro sensitivity data for chemo therapeutic drug, vincristine (particularly cell line-specific IC₅₀ values for 530 cell lines), from cancerrx (database of genomics of drug sensitivity), and cell multi-omics characteristics, from Cancer Cell Line Encyclopedia (CCLE) were used as inputs for the stratification algorithm. MDR transporter genes amplification, particularly ABCB4, ATP-binding cassette sub-family B member 4 (MDR3), amplification and ABCB1, ATP-binding cassette sub-family B member 1 (MDR1), amplification was used as a query of interest.

The panel of cell lines selected, according to various embodiments previously described herein, may be used for assaying drug transporter effect on a drug of interest, for example: anti-cancer drug. In an embodiment, representative cell lines are selected for each MDR transporter genes, with amplification (CAN/mRNA high) for a specific transporter with other MDR transporters in their wild-type condition. The cellular availability of an anti-cancer agent is assayed on these cell lines and with the addition of drug targeting specific transporters, to understand the effect of drug transporters on drug efflux.

The embodiments herein generate response curves (using inverse IC₅₀ values) between two populations; i.e., cell line population with altered (Amplified transporter of interest) v/s cell line population with wild type (status of transporter of interest) which is used in identification of drug transporter alterations. The alterations in drug transporter are correlated with loss of efficacy of the drug of interest which may be due to increased drug efflux. In an embodiment, response curves for cell line population with amplified ABCB4, versus wild type ABCB4 showed significant difference in median between the response curves indicating lesser efficacy in population with transporter amplification. In another embodiment, response curves for cell line population with amplified ABCB1, versus wild type ABCB1 showed significant difference in median between the response curves indicating lesser efficacy in population with transporter amplification.

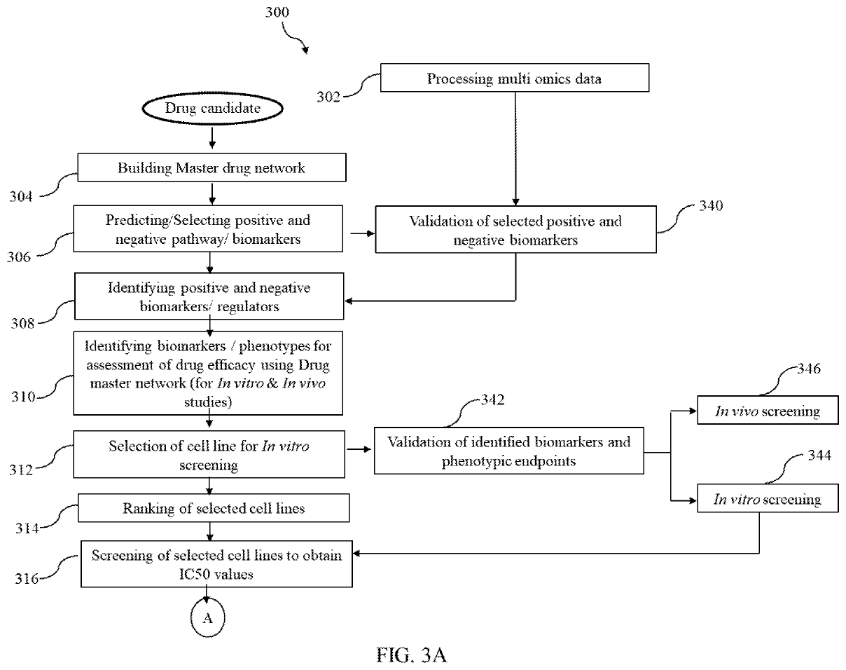

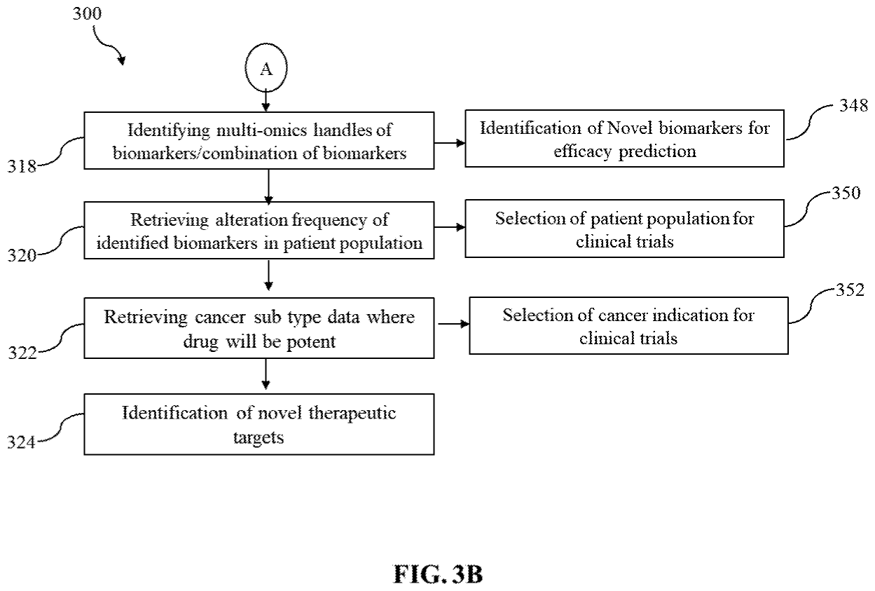

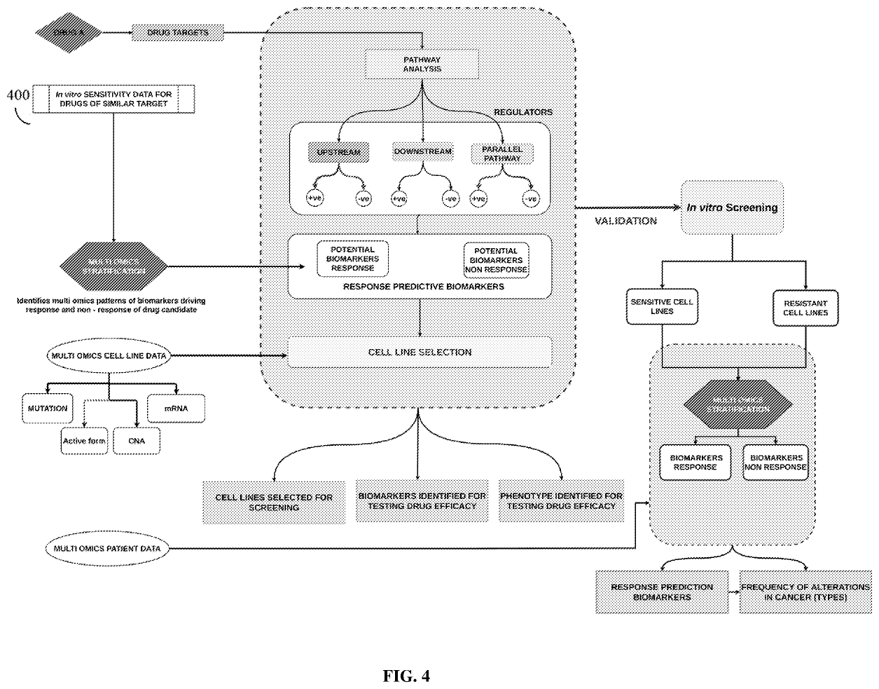

FIGS. 3A and 3B are schematic representations depicting overall process of a method 300 for identification, selection and validation of Human Cancer Cell lines and Biomarkers, according to embodiments as disclosed herein. FIG. 4 is a schematic representation of an example flow chart depicting a method 400 for drug discovery and development for identification, selection and validation of Cancer Cell lines and biomarkers, selection of patients in clinical trials, and for biomarkers, based on Pharmacological Target(s), according to an embodiment of the present invention.

In an embodiment herein, the method 300 for identification, selection and validation of human cancer cell lines and biomarkers based on pharmacological targets, cell line or patient multi-omics data is processed 302. The raw cell line/patient multi-omics data 302 can be obtained from various sources and in-house data. The obtained cell line/patient multi-omics data is processed using a novel solution for variant calling and assessment of functional impact of mutations. The processed mutation, copy number alterations, mRNA and active form data is then stored in an in-house cell line database and a patient database, which may be continuously updated with new data generated in-house.

The method 300 for identification, selection and validation of Cell lines and biomarkers, further includes constructing a ‘Master Drug Network’ around the pharmacological target(s) with pathway components 304 (enzymes and transcription factors). The pharmacological target, and Master drug network, according to embodiments herein, is based on drug candidate or drug of interest. In an example herein, the pathway components can be upstream regulators, downstream effectors, effectors regulated by bypass mechanisms and pathway cross talks. These pathway regulators and effectors are either tagged as positive biomarkers or negative biomarkers based on their effect on activation (upstream) or activity (downstream) of pharmacological target. In an example herein, the central nodes of the pathway are activated (over-expressed or aberrantly expressed) pharmacological target(s). Further, the sensitive and resistance pathways and regulators associated with a pharmacological target(s) are predicted/selected using the master drug network 306. Thereafter, the biomarkers are validated 340 based on alterations in pharmacological (inhibitor/drug) target, positive biomarker(s) and negative biomarkers(s) to identify specific biomarkers 308 useful in cell line selection. Once the biomarkers are predicted/selected 306, the biomarkers may be validated 340 based on alteration frequency in cancer (cell line and patient population), and impact of biomarker on survival (patient population). In an embodiment, the selected biomarkers are validated using in house (patient and/or cell line) database obtained by processing multi-omics data 302. The validated biomarkers and drug target/s are the basis for cell line selection, for in vitro screening. In an embodiment herein, validation of biomarkers are based on alteration frequency in cancer, functional impact on activation/activity of drug target gene(s), its impact on survival (survival data of patient population), efflux transporters and drug metabolizing enzymes which determine intracellular pharmacokinetics (PK) and drug resistance. The prediction and/or selection of biomarkers 306 are based on different level of regulators or pathway effectors (such as upstream, downstream, bypass, cross talks, and so on). In an example herein, the pathway will constitute well defined upstream, downstream, bypass mechanisms and pathway cross talks, and determinants of Pharmacokinetic resistance (efflux transporters and drug metabolizing enzymes). In an embodiment herein, regulators include upstream regulators comprising regulators which control activation of pharmacological target(s), both positively and negatively. The end point biomarkers of upstream pathways are activated pharmacological target(s). In an embodiment herein, regulators include downstream regulators, particularly of genes/pathways regulated by pharmacological target(s), positively/negatively, that function as effector in transducing signaling from drug target gene(s) to phenotypic end points. In an embodiment herein, bypass mechanisms include effector (active form) which perform same role as drug target(s) (active form) having same downstream target. In an embodiment herein, the pathway cross talks include biological pathways/effectors which regulate activation of the pharmacological target (upstream) or activity of pharmacological target (downstream). In an embodiment herein, the determinants of Pharmacokinetic resistance (PK) can include efflux transporters and pumps and drug metabolizing enzymes.

In an embodiment herein, as shown in FIG. 3A, the identification of positive and negative biomarkers and PK determinants of resistance for drug 308, by the drug master network analysis is based on different level of regulators (such as upstream, downstream, bypass, cross talks, and so on). In an example herein, different regulators are selected which could impact the drug efficacy in positive or negative manner. Table 1 shows the upstream regulator/biomarker impact on drug target and on drug efficacy. Table 2 shows the downstream regulator/biomarker impact on drug target and on drug efficacy. Table 3 shows the bypass activation/biomarker impact on drug target and on drug efficacy.

TABLE 1
Upstream regulator/biomarker impact
on drug target and on drug efficacy.

| | | Biomarker | Biomarker |
|---|---|---|---|
| | | impact | impact on |
| | | on drug target - | drug |
| Regulator | Alteration | activated form | efficacy |
| Upstream | Copy number gain/gain of | Activated drug | Positive |
| positive | function mutation/increased | target (s) -High | |
| regulator | mRNA expression etc | | |
| Upstream | Copy number loss/loss of | Activated drug | Negative |
| positive | function mutation/reduced | target (s) -Low | |
| regulator | mRNA expression etc | | |
| Upstream | Copy number loss/loss of | Activated drug | Positive |
| negative | function mutation/reduced | target (s) -High | |
| regulator | mRNA expression etc | | |
| Upstream | Copy number gain/gain of | Activated drug | Negative |
| negative | function mutation/increased | target (s) -Low | |
| regulator | mRNA expression etc | | |

TABLE 2
Downstream regulator/biomarker impact
on drug target and on drug efficacy

| | | Biomarker | Biomarker |
|---|---|---|---|
| | | impact on | impact on |
| | | drug target - | drug |
| Regulator | Alteration | activity | efficacy |
| Downstream gene | Constitutive | Disease | Negative |
| | activation | independent of | |
| | | drug target | |
| Downstream gene - | | | Negative |
| Negative regulation | | | |
| of tumor promoter | | | |
| downstream gene by | | | |
| drug target gene(s) | | | |
| Downstream gene -enhance | | | Positive |
| activity, still controlled | | | |
| by drug target gene(s) | | | |

TABLE 3
Bypass activation/biomarker impact
on drug target and on drug efficacy

| | | | Biomarker |
|---|---|---|---|
| | | Biomarker | impact on |
| | | impact on | drug |
| Regulator | Alteration | drug target | efficacy |
| Positive | Copy number gain/gain of | Disease | Negative |
| regulator of by- | function mutation/increased | independent | |
| pass regulation | mRNA expression etc | of drug target | |
| Negative | Copy number loss/loss of | | Positive |
| regulator of by- | function mutation/reduced | | |
| pass regulation | mRNA expression etc | | |

In an embodiment, the drug master network is used to identify biomarkers and phenotypes for assessment of drug efficacy post treatment during in vitro studies and at patient level during in vivo studies 310. In an embodiment, the biomarkers are identified for assessment of drug efficacy post treatment during in vitro studies. The biomarkers at multiple levels are provided from immediate downstream target of drug target gene(s), to biomarkers related to phenotypic end point/s. The phenotypic end points related to pharmacological target(s) are also provided which help in assessment of drug efficacy and validation at in vitro screening level. In an embodiment herein, the biomarkers (blood/tumor/CTC) are identified for assessment of drug efficacy post treatment at patient level during in vivo studies. Based on the drug mechanism of action, biomarkers from blood/tumor/CTCs are provided which will help in assessment of drug effect post treatment, in patients. Validation of identified biomarker 312 may be performed at in vitro and in vivo levels. In an embodiment herein, the identified biomarkers are validated during in vitro studies 344. In another embodiment herein, the identified biomarkers are validated during in vivo animal studies 346.

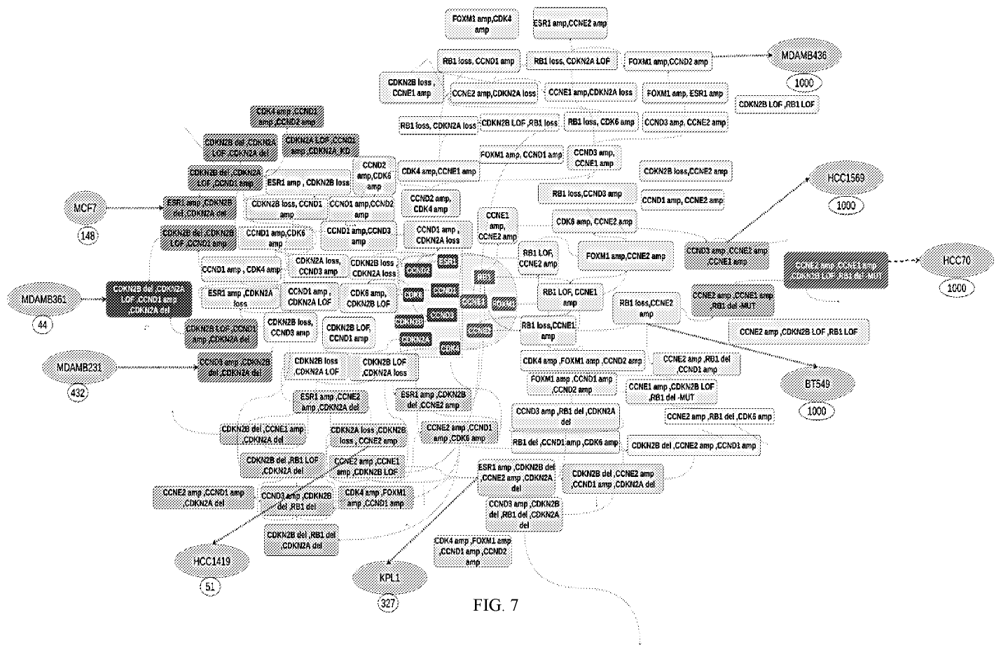

In an embodiment herein, the cell line or cell line subset is selected 312 based on alterations of validated biomarkers 340, for in vitro screening 326. The alteration patterns in genes of interest and few representative cell lines selected for each pattern is illustrated in FIG. 7. In an example herein, the cell line subset can be from available commercial cell lines/new cell lines. Further, the cell lines selected are ranked in order 314, i.e. ones with enhanced alterations of positive biomarkers (which would lead to drug sensitivity), followed by cell lines with alterations of both positive and negative biomarkers, and cell lines with enhanced alterations of negative biomarkers (which would lead to drug resistance). Cell line subset is selected, particularly to demonstrate the effectiveness of the compound of interest, identify possible resistance loops that can build in, validate various hypothesis built during pathway analysis, identify indirect/novel mechanisms for compound sensitivity/resistance. The identified subset of cell lines is a representation of global cohort (all cell lines) in terms of alteration of all genes (part of query) and patterns of alterations of gene (combination of alterations) related to the pathway. Further the selected cell lines are screened to obtain cell line specific IC₅₀ values 316. In an embodiment herein, after the selection of positive and negative biomarkers 306, followed by the validation of selected biomarkers 340, in vitro screening 344 is performed for drug response, and validation of biomarkers and phenotypic endpoints 342. In an embodiment herein, after the selection/identification of positive and negative biomarkers 306, followed by the identification of biomarkers for assessment of drug efficacy 310, in vivo screening 346 is performed for validation of biomarkers and phenotypic endpoints. In another embodiment herein, after the selection of positive and negative biomarkers 306 followed by the validation of selected biomarkers 340, selection of cell line 312 is performed for validation of identified biomarkers and phenotypic endpoints 342 by in vitro screening 344.

In an embodiment herein, following the in vitro screening of inhibitor compounds and drug of interest on the selected cell lines, the method 300 uses the cell line specific IC₅₀ values in identifying statistically significant multi-omics de-regulation patterns of biomarker(s) or a combination of biomarkers 318 responsible for enhancing sensitivity or resistance of cancer cell lines to the compounds or drug of interest. In an embodiment, the identification of multi-omics handles is performed by using novel stratification solution. In an embodiment herein, the method 300 uses the cell line specific IC₅₀ values to identify multi-omics handles of biomarkers or a combination of biomarkers which separates sensitive cell lines from resistant ones. These help to validate hypothesized biomarkers and identify novel biomarkers 348 for efficacy prediction and help to understand drug mechanism of actions.

Further in an embodiment herein, the method 300 is used to retrieve the alteration frequency of identified biomarkers in patient population 320. In an embodiment, the retrieval of alteration frequency of identified biomarkers in patient population 320 is used in selection of patient population for clinical trials 350. Further in an embodiment herein, the method 300 is used to retrieve the cancer sub type data where the drug of interest will be potent 322, based on alteration frequency and functional impact of biomarkers. In an embodiment, the retrieval of cancer subtype data 320 is used in selection of cancer indication for clinical trials 352. In an embodiment herein, the method 300 further includes identification of novel therapeutic target 324 based on identification of multiple level biomarkers and phenotypes.

Embodiments herein also include a system for performing the activities as disclosed in various embodiments of the method herein. In an embodiment, the system comprises processor and memory units for processing multi-omics data 302 to generate a cell line and patient database; for building a Master drug network 304 around pharmacological target including pathway players such as upstream regulators, downstream effectors, genes regulating by pass mechanisms and pathway cross talks; for selection of biomarkers 306 using pathway analysis of master drug network; for assessing biomarkers using multi-omics line data; for identifying biomarkers and/or phenotypes for assessment of drug efficacy 310 using a Drug master network; for selecting cell line subset 312; for screening cell lines 316 to obtain cell line IC₅₀ values; for receiving the IC₅₀ values and identifying multi-omics handles of biomarkers or combination of biomarkers 318 which separate sensitive cell lines from resistant ones; for selection of patient and/or cancer subtype for clinical trial based on alteration frequency of identified biomarkers in patient population and/or cancer sub-type data, according to embodiments herein.

Embodiments herein also include a method for identification of suitable treatment, with a suitable drug or drug combination. Various drugs and drug combinations, generally known in the art may be used, which would be apparent to a skilled practitioner. The embodiments herein may be used to facilitate or assist patient's physician in prescribing suitable therapy. The method, according to embodiments herein, includes integration of multi-omics data from cancer patients, pharmacokinetic data from patients, ex vivo (patient primary cell lines, organoids, tumors) and in vivo models of cancer (with patient tumors/cells), and assessment to identify suitable therapeutic approach. The method further includes classification of individual patients, in terms of Drug Master networks.

Embodiments are further described herein by reference to the following examples by way of illustration only and should not be construed to limit the scope of the claims provided herewith.

### Example 1: Case Study

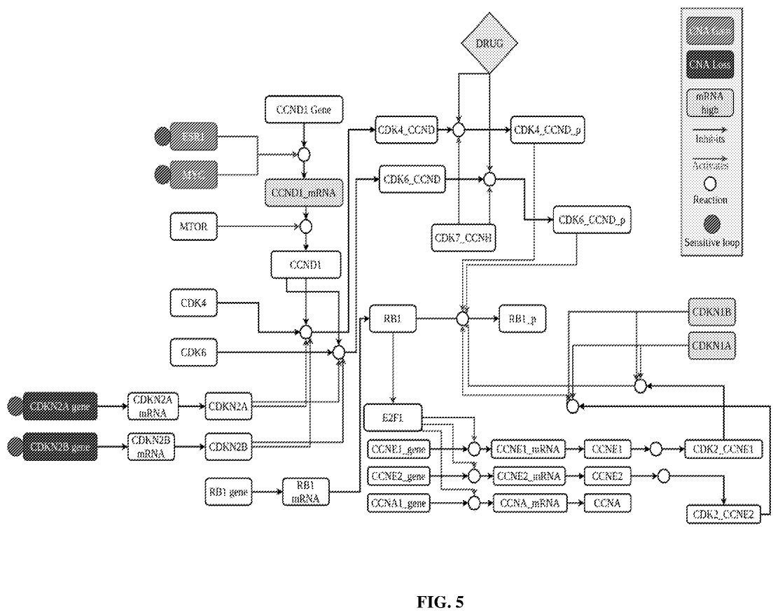

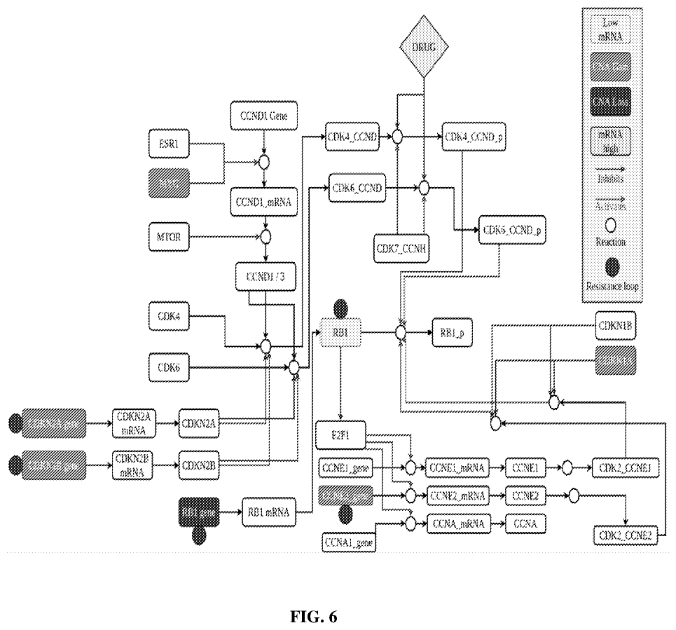

Palbociclib, a targeted inhibitor of CDK4/6 indicated for breast cancer. Identification of Human Breast Cancer cell lines sensitive and resistant to Palbociclib using the method 300 for drug discovery and development, particularly for identification, selection and validation of Human Cancer Cell lines and Biomarkers based on Pharmacological Target(s). A comparison of Predicted Efficacy with Observed Efficacy in cell lines/patients is provided. The pharmacological target is Ser/Thr-kinase component of cyclin D-CDK4/6, which inhibits CDK4/6 phosphorylation of retinoblastoma (RB) protein family (RB1). The phenotypic end point of which is the inhibition of cell proliferation. The regulators of CDK4/6 in cells are identified as shown in Table 4. Further, the Breast Cancer Cell Lines are selected based on the multi-omics and pathway regulator status. Table 5 shows the cell lines predicted to be sensitive to Palbociclib. FIG. 7 shows the alteration patterns in genes of interest and few representative cell lines selected for each pattern. FIG. 5 shows an example diagram of MCF7 breast cancer cell line, a cell lines predicted to be sensitive to Palbociclib. FIG. 6 shows an example diagram of HCC2157 breast cancer cell line, a cell line predicted to be resistant to Palbociclib. The predicted response (sensitivity or resistance to Palbociclib) was compared with cell line in vitro potency data (IC₅₀) of Palbociclib. The predicted responses were in agreement with observed data (IC₅₀) for Palbociclib's activity on various Human Breast Cancer cell lines. Table 6a and 6b show the comparison of predicted response values, for both sensitive and resistant to Palbociclib, with observed data.

TABLE 4
Identification of regulators of CDK4/6 in Cells

| S. No. | GENE | DESCRIPTION | DEREGULATION |
|---|---|---|---|
| 1 | CCND1 | Drug Target | Amplification/upstream |
| | | | upregulation |
| 2 | CDK4 | Drug Target | Amplification/upstream |
| | | | upregulation |
| 3 | CDK6 | Drug Target | Amplification/upstream |
| | | | upregulation |
| 4 | CDKN2A | Inhibitors of drug target | Deep del/LOF mutation |
| 5 | CDKN2B | Inhibitors of drug target | Deep del/LOF mutation |
| 6 | CDKN2C | Inhibitors of drug target | Deep del/LOF mutation |
| 7 | MYC | Transcription factor of CCND1 | Amplification/upstream |
| | | | upregulation |
| 8 | E2F1 | Downstream effector | Amplification/upstream |
| | | | upregulation |
| 9 | ESR1 | Transcription factor of CCND1 | Amplification/GOF mutation |
| 10 | CDK7 | Activator of drug target | |
| 11 | CCNH | Activator of drug target | |
| 12 | CDK2 | Activator of next phase of cell | Amplification/upstream |
| | | cycle (post S phase) | upregulation |
| 13 | CCNB1 | Activator of next phase of cell | Amplification/upstream |
| | | cycle (M phase) | upregulation |
| 14 | CDK1 | Activator of next phase of cell | Amplification/upstream |
| | | cycle (M phase) | upregulation |
| 15 | CDKN1A/B/C | Inhibitor of cyclin - CDK | |
| | | complex, activating post S | |
| | | phase | |
| 16 | RB1 | Important downstream effector | Deep del/LOF mutation |
| 17 | FZR1 | Regulator of drug target | Deep del/LOF mutation |
| | | degradation | |
| 18 | TP53 | Transcription factor of p21 | Deep del/LOF mutation |
| 19 | RB1 | Important downstream effector | Deep del/LOF mutation |
| 20 | FZR1 | Regulator of drug target | Deep del/LOF mutation |
| | | degradation | |
| 21 | TP53 | Transcription factor of p21 | Deep del/LOF mutation |
| 22 | WEE1 | G2 - M check point regulators | |
| 23 | CHEK1 | G2 - M check point regulators | |
| 24 | FOXM1 | Transcription regulator of | Amplification/upstream |
| | | cyclins regulating next phase of | upregulation |
| | | cell cycle (post S) | |
| 25 | TK1 | Transcriptional target of E2F1 | Amplification/upstream |
| | | | upregulation |

TABLE 5
Cell lines predicted to be sensitive to Palbociclib

| | | Breast cancer sub | |
|---|---|---|---|
| S. No. | Cell Line | type | Characteristics |
| 1 | MCF7 | ER +ve, HER2 | ESR1 amp, MYC amp, CDKN2A del, |
| | | Normal | CDKN2B del, RB1 WT |
| 2 | MDAMB361T | ER +ve, HER2 | CCND1 amp, CDKN2A LOF mut, |
| | | Amplified | CDKN2B del, RB1 WT |
| 3 | HCC1500 | ER +ve, HER2 | CCND1 amp, CDKN2A del, CDKN2B |
| | | Normal | del, MYC amp, RB1 WT |
| 4 | MDAMB231 | ER −ve, HER2 | CDKN2A del, CDKN2B del, CCND3 |
| | | Normal | amp, RB1 WT |
| 5 | EFM19 | ER +ve, HER2 | CDKN2A del, CDKN2B del, RB1 WT |
| | | Normal | |
| 6 | HCC2157 | ER −ve, HER2 −ve | RB1 del, CDKN2A amp, CDKN2B amp, |
| | | | CCNE2 amp |
| 7 | HCC1569 | ER −ve, HER2 | E2F1 amp, CCNE1 amp, CCNE2 amp, |
| | | Amplified | RB1 WT |
| 8 | MDAMB157 | ER −ve , HER2 | RB1 del, CCNE1 amp, CDKN1B del |
| | | Normal | |
| 9 | DU4475 | ER −ve, HER2 | RB1 del |
| | | Normal | |
| 10 | MDAMB468 | ER −ve, HER2 | RB1 del |
| | | Normal | |

Amp—amplification, del—deep deletion, LOF—Loss of function mutation, WT—wild type.

TABLE 6a
Comparison of Predicted Values (sensitive
to Palbociclib) with Observed Data.

| | | Predicted Response | Observed IC₅₀ |
|---|---|---|---|
| S. No. | Cell Line | (sensitive to Palbociclib) | (μM) |
| 1 | MCF7 | RESPONSIVE | 0.148 |
| 2 | MDAMB361 | RESPONSIVE | 0.044 |
| 3 | HCC1500 | RESPONSIVE | 0.045 |
| 4 | MDAMB231 | RESPONSIVE | 0.432 |
| 5 | EFM19 | RESPONSIVE | 0.027 |

TABLE 6b
Comparison of Predicted Values (Resistant
to Palbociclib) with Observed Data

| | | Predicted Response | Observed IC₅₀ |
|---|---|---|---|
| S. No. | Cell Line | (Resistant to Palbociclib) | (μM) |
| 1 | HCC2157 | NON - RESPONSIVE | >1 |
| 2 | HCC1569 | NON - RESPONSIVE | >1 |
| 3 | MDAMB157 | NON - RESPONSIVE | >1 |
| 4 | DU4475 | NON - RESPONSIVE | >1 |
| 5 | MDAMB468 | NON - RESPONSIVE | >1 |

Embodiments disclosed herein add value both at early discovery (lead identification and optimization) and late (preclinical and clinical development) stages to maximize the success rate of candidate drug, and also helps in reducing the cost and time during drug development and personalized medicine. Embodiments disclosed herein selects indication(s), with high frequency of alterations of positive biomarkers which drive/enhance drug sensitivity (based on patient population data), where the candidate drug molecule can have high efficacy. Embodiments disclosed herein provide multi-omics alteration of biomarker/combination of biomarkers, which can be used to select specific cohort of patients where the drug can respond with high efficacy. Embodiments disclosed herein selects lesser number of cell lines for screening (yet helps to understand drug mechanism of action in depth). This helps to avoid high throughput screening on larger set of cell lines at high cost. Embodiments disclosed herein reduces time taken for screening cell lines as the number of cell lines is lesser than the number selected by empirical selection. Embodiments herein achieve drug response prediction in selected cell line. Embodiments herein are useful in identification of novel biomarkers and therapeutic targets, particularly for selection of drug combinations to maximize clinical response. It is understood that various modifications and alterations to the disclosed embodiments of protein extraction would be apparent to a person skilled in the art without departing from the scope of the embodiments disclosed herein.

### Example 2: Biomarker Selection

Valid biomarkers were identified for the assessment of efficacy of the candidate drugs. The platform used to identify valid biomarkers is the system 300. By building drug master networks around the pharmacological target of interest, direct and indirect downstream effectors of pharmacological drug targets are selected as biomarkers in order to efficiently assess efficacy of candidate drugs. Downstream effector molecules that bind to the drug target and are directly regulated by the drug are known as downstream effectors. Further, indirect effectors are molecules that are regulated by the drug target through other intermediate molecules. Any alteration in the activity of the pharmacological drug target can either positively or negatively affects the activity or function of the effector molecule. Downstream effectors of drug targets that regulate phenotypic endpoints are selected as appropriate phenotypes that are subsequently used to assess drug efficacy. The system 300 can identify biomarkers for the assessment of efficacy of Cyclin Dependent Kinase 4/6 (CDK4/6) inhibitors.

Results/Observations: The system 300 can identify both direct and indirect biomarkers by building a drug master network around the pharmacological target of interest. The appropriate phenotype that was selected was that of proliferation. The biomarker linked to the proliferative phenotype is the transcriptionally active E2F1. Biomolecule CDK4/6 inhibits retinoblastoma 1 (RB1) by phosphorylation. Non phosphorylated RB1 binds and thereby, inhibits E2F1. Free E2F1 facilitates the G1-S phase transition of the cell cycle, thereby promoting cell proliferation. The direct biomarker selected is a hyper-phosphorylated RB1 which will decrease with drug treatment. The indirect biomarkers selected include TK1, Activated-cyclin A/E bound to phosphorylated CDK2, and Phosphorylated Forkhead box protein M1 (FOXM1).

TABLE 7
Selection of direct biomarkers

| | | Expected trend | |
|---|---|---|---|
| | | with drug | |
| Biomarker | Role | treatment | Biomarker |
| RB1 hyper | Drug target CDK4/6 | RB1 (non | RB1 hyper |
| phosphorylated | phosphorylates RB1 | phosphorylated) - | phosphorylated |
| form | and inactivates it | Increase | form |

TABLE 8
Selection of indirect biomarkers

| | | Expected trend | |
|---|---|---|---|
| | | with drug | |
| Biomarker | Role | treatment | Biomarker |
| TK1 expression | Transcriptional | Decrease | TK1 expression |
| | target of E2F1 | | |
| | which is negatively | | |
| | regulated by RB1 | | |
| Activated cyclin A/E | Transcriptional | Decrease | Activated cyclin A/E |
| bound CDK2 | target of E2F1, | | bound CDK2 |
| (phosphorylated | activated during S | | (phosphorylated |
| form) | phase of cell cycle | | form) |
| Phosphorylated | Transcriptional | Decrease | Phosphorylated |
| FOXM1 | target of E2F1, | | FOXM1 |
| | activated during G2 - | | |
| | M phase of cell | | |
| | cycle | | |

TABLE 9
Selection of appropriate phenotype

| Phenotype | Linked Biomarker | Rationale |
|---|---|---|
| Proliferation | Transcriptionally | CDK4/6 (inhibitory |
| | active | phosphorylation) RB1 |
| | E2F1 | RB1 (binds and inhibits) |
| | | E2F1 |
| | | E2F1 → G1 - S phase of cell |
| | | cycle transition → Cell |
| | | cycle (proliferation) |

### Example 3

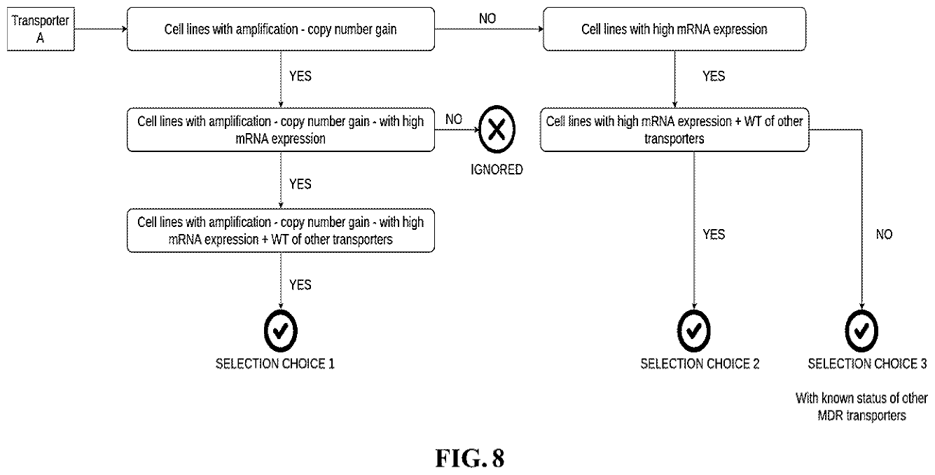

Embodiments herein select a panel of cell lines for assaying the effect of drug transporters on anti-cancer agents. In order to assess the effect of drug transporters on the efflux of drugs, parameters such as cellular availability of the anti-cancer drug is assayed on the selected cell lines along with drugs targeting specific transporters. FIG. 8 is a flowchart representation of the method of selection of the cell lines.

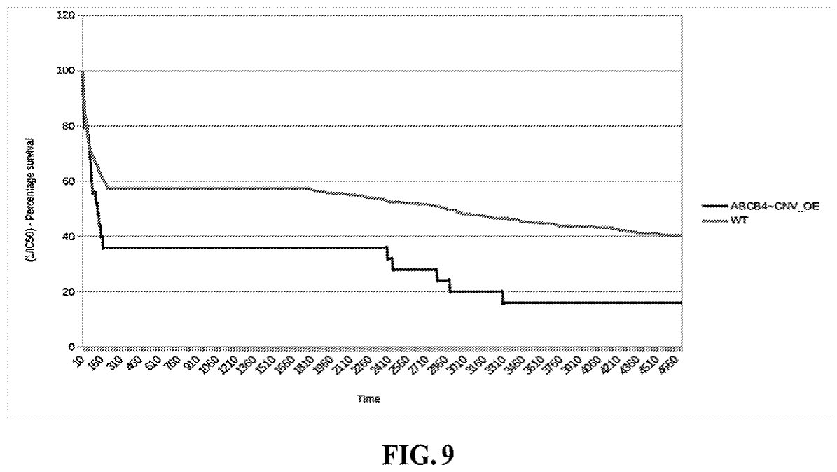

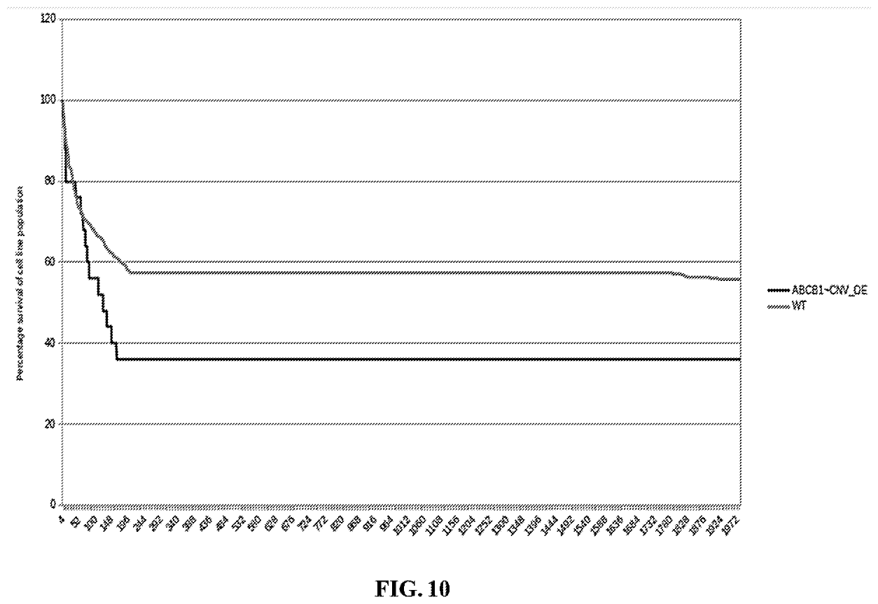

Results/Observations: The inputs for the system 100 include in vitro sensitivity data such as cell-line specific IC₅₀ values for 530 cell lines, for the chemotherapeutic drug vincristine from Cancerrx (database of genomics of drug sensitivity) and cell multi-omics characteristics from CCLE (Cancer Cell Line Encyclopaedia project). Amplified MDR Transporter genes, ABCB4-Amplification (ATP-binding cassette sub-family B member 4 Amplification) and ABCB1-Amplification (ATP-binding cassette sub-family B member 1 Amplification) were queries of interest. Embodiments herein compare the cell line populations with altered drug transporters with cell line populations with wildtype drug transporters in order to generate response curves using IC50 values. The response curves for the two populations were seen to have significant differences in median to indicate amplification of transporter. Significant loss in efficacy of the drug is also seen because of an increase in the efflux of the drug. FIG. 9 is a graphical representation of response curves depicting loss of efficacy of the drug in ABCB4 Amplified condition compared to wildtype conditions as predicted by embodiments herein. It is seen that there is a significant difference in the median value between the response curves that was plotted for two cell line populations that indicate a lower efficacy in the cell line population with transporter amplification. This result is validated by previous relevant findings in the field, which discloses that an increased resistance to the drug vincristine is seen in conditions where ABCB4 transporter is amplified. FIG. 10 is a graphical representation of the response curves for the cell line population with amplified ABCB1 conditions as compared to the wildtype condition. It is seen that there is a significant difference in the median between the response curves plotted for the two cell line populations that indicate lesser efficacy in population with the ABCB1 transporter amplification condition. This result is also validated by previous relevant findings in the field, which discloses an increased resistance to the drug vincristine is seen in conditions where ABCB1 transporter is amplified. Table 10 shows the gene of interest, CNA status and mRNA z scores for the selected cell lines.

TABLE 10
Gene of interest, CAN Status and mRNA
z scores for cell lines selected.

| | | | | mRNA - z score |
|---|---|---|---|---|
| | | Gene of | | (wrt diploid |
| S. No | Cell Line Selected | interest | CNA status | samples) |
| 1 | SNU878 | ABCB11 | Amp (OE) | 32.3165 |
| 2 | MOLP2 | ABCC10 | Amp (OE) | 4.8581 |
| 3 | YAPC | ABCC10 | WT | 12.1496 |
| 4 | CALU6 | ABCC4 | Amp (OE) | 2.2544 |
| 5 | HS172T | ABCC4 | WT | 8.6999 |
| 6 | ABC1 | ABCC5 | Amp (OE) | 5.1993 |
| 7 | FU97 | ABCC6 | WT | 6.1299 |
| 8 | SW948 | ABCB1 | WT | 7.5105 |
| 9 | SKMEL31 | ABCB4 | WT | 8.398 |
| 10 | BC3C | ABCC1 | WT | 7.6491 |
| 11 | HUH1 | ABCC2 | WT | 11.6513 |
| 12 | MDAMB453 | ABCC11 | WT | 8.4271 |
| 13 | KYM1 | ABCC12 | WT | 16.3688 |

### Example 4

Patient and indication selection. With the input of in vitro efficacy data, the stratification algorithm identifies favorable and unfavorable multi-omics characteristics such as mutations, copy number variations (CNVs) and mRNA deregulations that are known to affect drug sensitivity or drug resistance characteristics. Embodiments herein can identify cancer types and/or subtypes in patients based on the favorable genomics, as is listed in the database. For the drug Palbociclib, a CDK4/6 inhibitor, deep deletion of CDKN2A and/or deep deletion of CDKN2B, along with a wild-type status of RB1 molecule as favorable genomics. Cancer cell lines from the cancer cell line encyclopedia were used as the global population for maximum efficacy while selecting and identifying individuals with cancer types based on favorable multi-omics.

Results/Observations: Embodiments herein select glioma/glioblastoma cells having cell line subsets with genomic alterations that play critical roles in drug responses. Table 11 depicts a list of genomic alterations and its favorability as determined by the system 300, along with the number of glioma cell lines that possess the specific alteration. Some other types of cancers that can be identified include melanoma, breast cancer, kidney cancer, head and neck cancers, and ovarian cancer.

TABLE 11
Classification of genomic alterations, and the
number of cell line with the genomic alteration

| | | | No of glioma |
|---|---|---|---|
| | | | cell lines |
| | | | possessing this |
| S. No. | Genomic aA2:C14 | FAVOURABILITY | alteration |
| 1 | CDKN2A Deletion | FAVOURABLE | 8 |
| 2 | CDKN2B Deletion | FAVOURABLE | 7 |
| 3 | CDKN2B Deletion, CDKN2A | FAVOURABLE | 7 |
| | Deletion | | |
| 4 | CCND1_Amplification | FAVOURABLE | 1 |
| 5 | CCND1_Amplification, | FAVOURABLE | 1 |
| | CDKN2A Deletion | | |
| 6 | CDKN2C Deletion | FAVOURABLE | 1 |
| 7 | CDKN2B Deletion, | FAVOURABLE | 1 |
| | CCND1_Amplification | | |
| 8 | CDKN2C Deletion, | FAVOURABLE | 1 |
| | CDKN2ADeletion | | |
| 9 | CDKN2B Deletion, | FAVOURABLE | 1 |
| | CCND1_Amplification, | | |
| | CDKN2A Deletion | | |
| 10 | RB1_Loss of function mutation | UNFAVOURABLE | 4 |
| 11 | RB1 Deletion | UNFAVOURABLE | 3 |
| 12 | RB1 Deletion, RB1_Loss of | UNFAVOURABLE | 2 |
| | function mutation | | |
| 13 | CDKN2B Deletion, RB1 | UNFAVOURABLE | 1 |
| | Deletion, CDKN2A Deletion | | |
| 14 | CDKN2B Deletion, RB1_Loss | UNFAVOURABLE | 1 |
| | of function mutation, CDKN2A | | |
| | Deletion | | |
| 15 | RB1 Deletion, CDKN2A | UNFAVOURABLE | 1 |
| | Deletion | | |
| 16 | CDKN2B Deletion, RB1 | UNFAVOURABLE | 1 |
| | Deletion | | |
| 17 | CDKN2B Deletion, RB1_Loss | UNFAVOURABLE | 1 |
| | of function mutation | | |
| 18 | RB1 Loss of function mutation, | UNFAVOURABLE | 1 |
| | CDKN2A Deletion | | |

### Example 5

The system 100 predicts the response of a patient to a particular drug or a combination of drugs based on the multi-omics of the patient. The system is trained for prediction of response to a number of different drugs using an in-house patient database. A retrospective dataset from acute myeloid leukaemia (AML) patients on standard Cytarabine and Daunorubicin treatment was used for response prediction training by system 100. Data from more than 400 patients with AML, on a treatment with Cytarabine and Daunorubicin was present in the in-house patient database. This dataset was used as input for training the system 100 which predicted the response of 10 AML patients to treatment by the two drugs.

Results/Observations: The predicted response was in accord with the actual response shown by the patients. Table 12 shows the actual response of the 10 AML patients as compared to that of the AI predicted response.

TABLE 12
Response predicted by the AI algorithm
as compared to the actual response

| | | Actual | Predicted |
|---|---|---|---|
| S. No. | Patient ID | Response | Response |
| 1 | TCGA-AB-2893 | RESPONDER | RESPONDER |
| 2 | TCGA-AB-2895 | RESPONDER | RESPONDER |
| 3 | TCGA-AB-2896 | RESPONDER | RESPONDER |
| 4 | TCGA-AB-2903 | RESPONDER | RESPONDER |
| 5 | TCGA-AB-2935 | RESPONDER | RESPONDER |
| 6 | TCGA-AB-2812 | NON-RESPONDER | NON-RESPONDER |
| 7 | TCGA-AB-2836 | NON-RESPONDER | NON-RESPONDER |
| 8 | TCGA-AB-2860 | NON-RESPONDER | NON-RESPONDER |
| 9 | TCGA-AB-2866 | NON-RESPONDER | NON-RESPONDER |
| 10 | TCGA-AB-2887 | NON-RESPONDER | NON-RESPONDER |

### Example 6: MDS Module 111

The tool uses the PDB (Protein Data Bank) protein structure to construct wild type and mutant models, and further builds activity projections for the same. Machine Learning (ML) gene models are created using pattern recognition protocol, which is in turn u sed to predict the mutation functionality.

BRAF (v-raf murine sarcoma viral oncogene homolog B1) is a serine/threonine protein kinase that plays a critical role in the RAS-RAF-MEK-ERK mitogen activated protein kinase (MAPK) cell signaling pathway. 83 mutations in the BRAF gene were used to create the BRAF model, which was used to then predict the functionality of the different mutation signatures of BRAF. 5 mutation signatures were predicted for functionality using the tool. The predicted outcome was validated by previous relevant findings in the field, for mutations. Table 13 shows the five mutation signatures along with the predicted and literature based functionality.

TABLE 13
Functionality prediction of 5 BRAF mutations using the MDS tool.

| | | Mutation | Predicted |
|---|---|---|---|
| S. No. | Protein | Signature | Functionality |
| 1 | BRAF | F595L | GOF |
| 2 | BRAF | G465A | GOF |
| 3 | BRAF | A728V | GOF |
| 4 | BRAF | G466A | GOF |
| 5 | BRAF | V600K | GOF |

### Example 7: Second Module 107

The NCCN Guidelines are a comprehensive set of guidelines detailing the sequential management decisions and interventions that currently apply to 97 percent of cancers affecting patients. The FDA also outlines pre-defined rules, based on which cancers are classified according to the risk of relapse, response, etc. Embodiments herein identify multi-omics signatures that can contribute to enhanced drug sensitivity or resistance and classifies the patient and/or cell lines into different subgroups or risk zones for treatment by a particular drug, or a combination of drug therapy and other categories based on pre-defined rules. Both cytogenetics and mutations determine the prognosis and response to standard chemotherapy treatment in patients with AML. Risk prediction, stratification, and assessment can help make an informed decision for treatment as prognosis can be predicted. Based on NCCN guidelines and European Leukaemia Net (ELN) recommendation, and based on genomic characteristics, patients with AML can be stratified into: Favourable risk; Intermediate risk and Adverse risk. Data from more than 4000 patients with AML, on a treatment with Cytarabine and Daunorubicin was present in the in-house patient database. Patient specific genomic characteristics and prognosis for patients treated with the standard chemotherapy were used as input to the system 100. The retrospective dataset of AML patients was used to predict the risk of 5 AML patients.

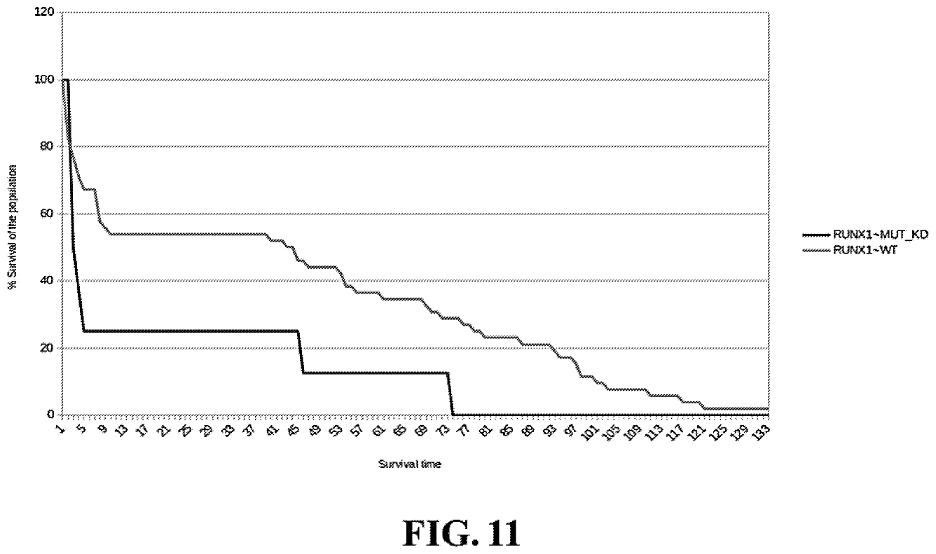

Results/Observations: Table 8 shows the risk prediction for the 5 AML patients as compared to the risk stratification that was done by genomic signatures. The predicted risk is seen to be in accord with the actual risk of the patients. Genomic data, survival of patients and other characteristics were obtained from the classification, and genomic markers which were found to be clinically relevant for either improved or poor response of the standard chemotherapy in patients were identified. The data was then used to generate survival curves. The median difference between survival curves of the altered cell line population as compared to the wild type population can be observed in FIGS. 11 to 14. The differences observed were found to be in agreement with that found in the literature evidence.

TABLE 14
Risk prediction of 5 AML patients from a retrospective dataset.

| | | | Genomic signature for risk |
|---|---|---|---|
| S. No. | Patient ID | Predicted risk zone | stratification |
| 1 | aml_ohsu_2018_10- | INTERMEDIATE | NPM1 wild type + FLT3 wild |
| | 00715 | | type |
| 2 | aml_ohsu_2018_13- | ADVANCED | ASXL1 mut |
| | 00237 | | |
| 3 | aml_ohsu_2018_13- | INTERMEDIATE | NPM1 WT + FLT3 WT |
| | 00250 | | |
| 4 | aml_ohsu_2018_13- | ADVANCED | RUNX1 mut |
| | 00515 | | |
| 5 | aml_ohsu_2018_13- | FAVORABLE | NPM1 mut + FLT3 WT |
| | 00535 | | |

### Example 8: Third Module 108

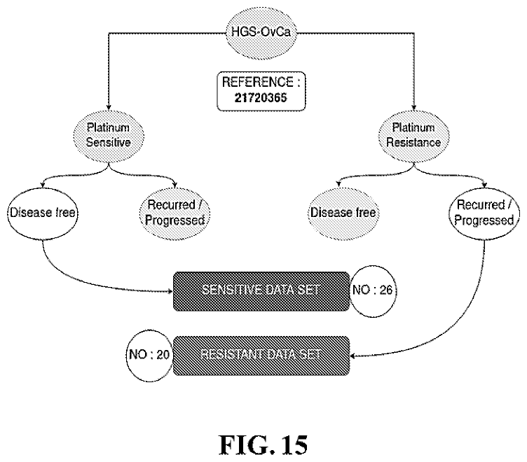

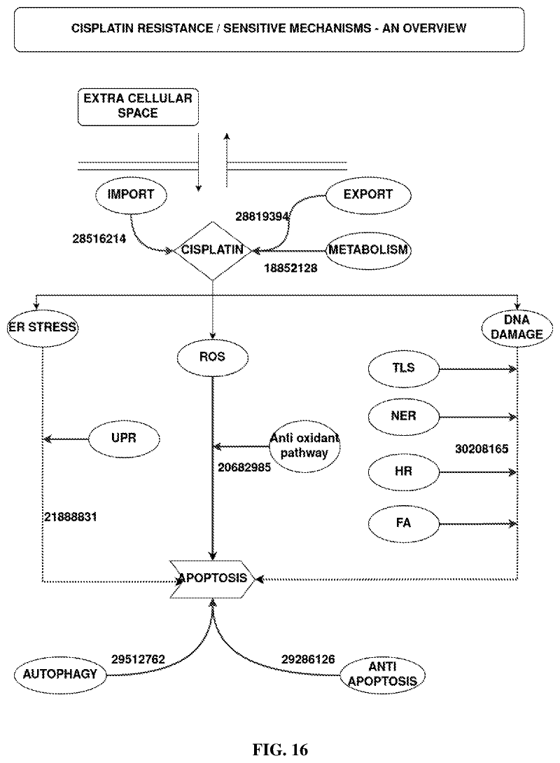

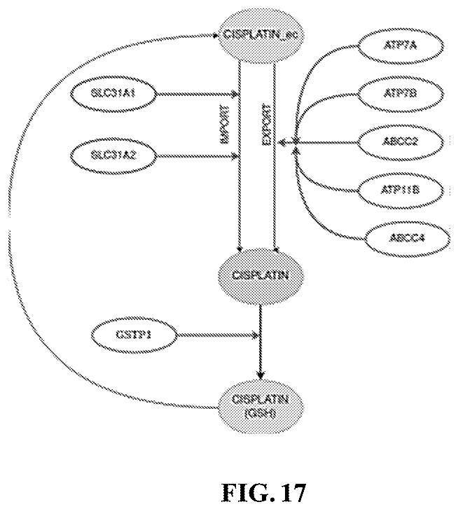

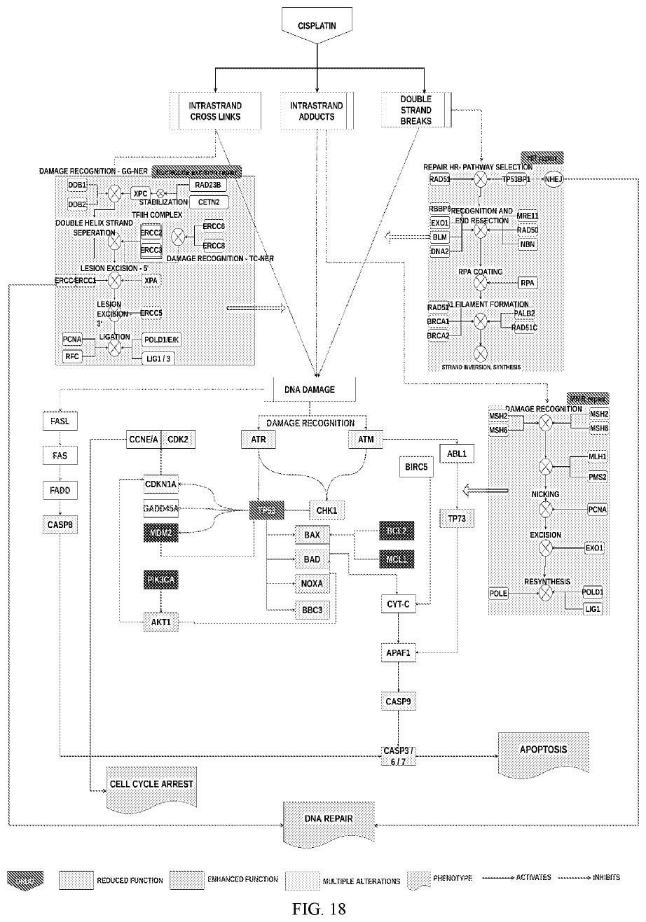

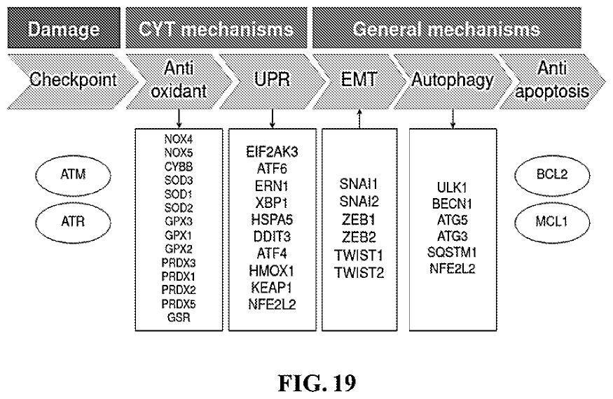

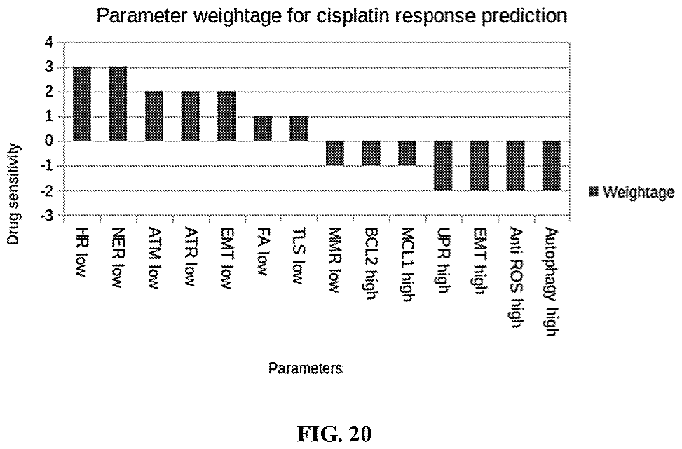

The system 100 is used to predict the response for platinum-based drug in ovarian serous carcinoma. 46 patients (26 responderspatients and 20 non-non-responders patients) with known responses to platinum agents were used to build a dataset. This dataset was then used to validate response scores as predicted by the response predictor. FIGS. 15 and 16 depict the resistance and sensitive mechanisms for platinum drug, Cisplatin. FIGS. 17 and 18 are schematic representations of the drug master network for the platinum based drug, Cisplatin. Other relevant pathways and biomarkers as identified by the system, according to embodiments herein, by the drug master network created for platinum-based drug Cisplatin are depicted in FIG. 19. All parameters such as biology of the drug/inhibitor's target, upstream and downstream regulators, influx and efflux mechanisms, metabolism, etc. are analyzed and given individual scores based on the indicated weightages. The weightages given for each parameter in order to predict response of patients to Cisplatin is shown in FIG. 20. A cumulative score of all parameters is then given by a scoring tool, called the Chemo sensitive score. This score is indicative of the response of a patient to the platinum agent cisplatin. Scores>1 were classified to be responders and others as non-responder patients.

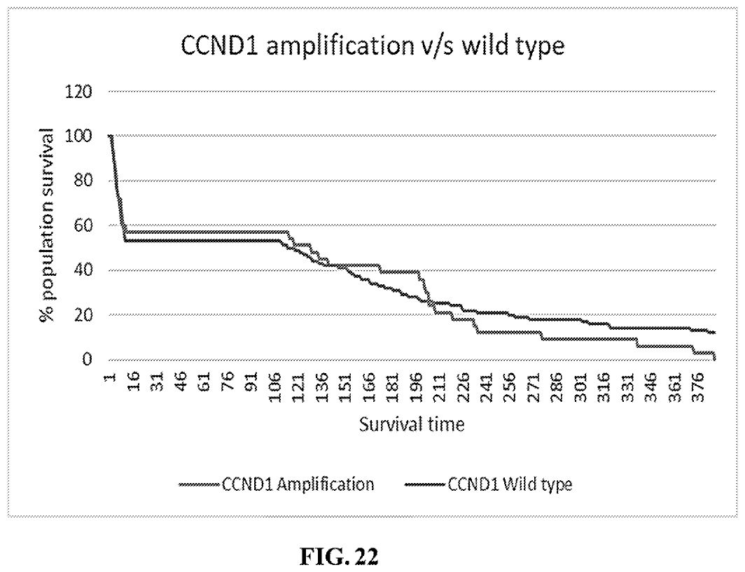

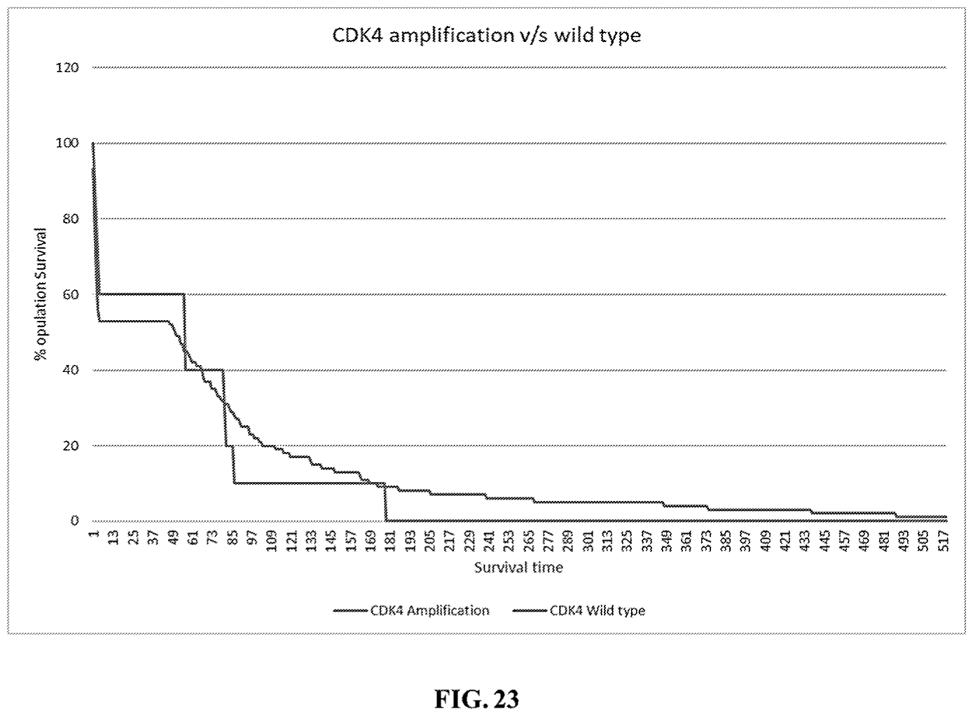

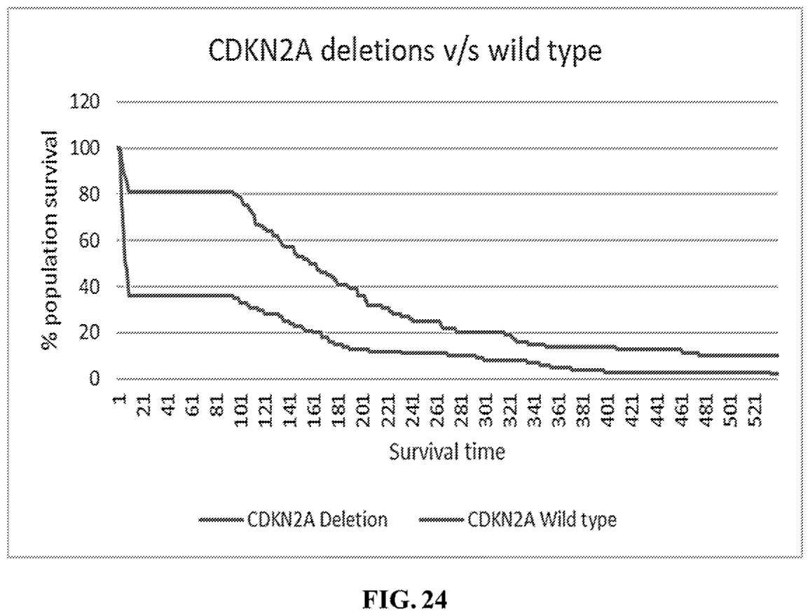

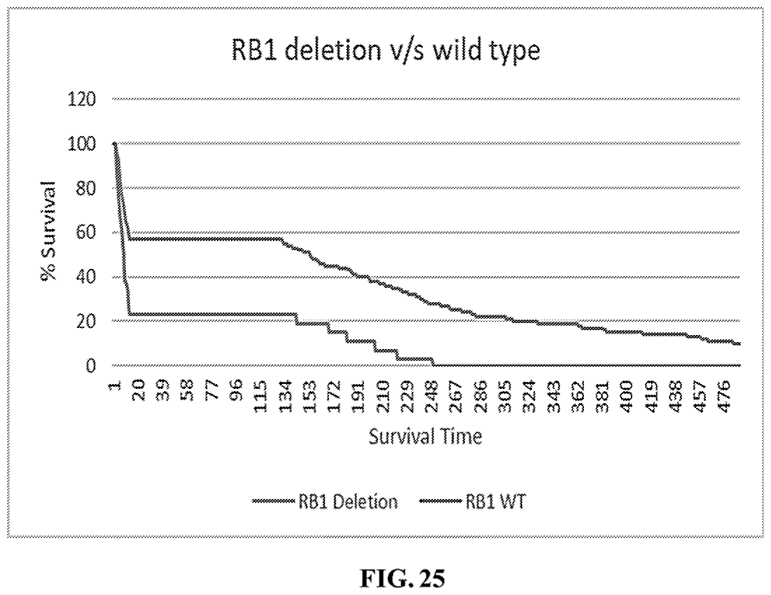

FIG. 21 is a schematic representation of the workflow for the multi-omics based stratification process. FIG. 22 is a graphical representation of predicted response curves of the cell line population with amplified CCND1 gene as compared to the cell line population with wildtype CCND1 gene for drug Palbociclib. FIG. 23 is a graphical representation of predicted response curves of the cell line population with amplified CDK4 gene as compared to the cell line population with CDK4 gene for drug Palbociclib. FIG. 24 is a graphical representation of predicted response curves of the cell line population with deleted CDKN2A gene as compared to the cell line population wildtype CDKN2A gene for drug Palbociclib. FIG. 25 is a graphical representation of the predicted response curves of the cell line population with deleted RB1 gene as compared to the cell line population wild type RB1 gene for drug Palbociclib.

Results/Observations: The predicted response when compared to that of the actual response of the patients, was found to be nearly 80% accurate.

Tables 15 and 16 show the lists of patients examined with cumulative scores and response as predicted both for responders and non-responderspatients by the response predictor as compared to validation dataset.

TABLE 15
List of patients examined with cumulative scores and
response as predicted for responder patients by the
response predictor as compared to validation dataset.

| | | Cumulative | Predicted | Clinical |
|---|---|---|---|---|
| S. No. | TCGA IDA2A2:C20 | score | Response | response |
| 1 | TCGA-04-1347 | 7 | R | R |
| 2 | TCGA-04-1367 | 6 | R | R |
| 3 | TCGA-09-2050 | 4 | R | R |
| 4 | TCGA-09-2053 | 3 | R | R |
| 5 | TCGA-09-2056 | 5 | R | R |
| 6 | TCGA-13-0762 | 7 | R | R |
| 7 | TCGA-13-0885 | 8 | R | R |
| 8 | TCGA-13-0886 | 3 | R | R |
| 9 | TCGA-13-0889 | 3 | R | R |
| 10 | TCGA-13-0890 | 7 | R | R |
| 11 | TCGA-13-0900 | 3 | R | R |
| 12 | TCGA-13-0905 | 5 | R | R |
| 13 | TCGA-13-0906 | 3 | R | R |
| 14 | TCGA-13-0910 | 2 | R | R |
| 15 | TCGA-13-0916 | 2 | R | R |
| 16 | TCGA-13-0919 | 2 | R | R |
| 17 | TCGA-13-1403 | 2 | R | R |
| 18 | TCGA-13-1481 | −2 | NR | R |
| 19 | TCGA-13-1492 | 6 | R | R |
| 20 | TCGA-13-1504 | 0 | NR | R |
| 21 | TCGA-23-1118 | 3 | R | R |
| 22 | TCGA-23-2078 | 3 | R | R |
| 23 | TCGA-25-2391 | 3 | R | R |
| 24 | TCGA-61-2092 | 2 | R | R |
| 25 | TCGA-61-2094 | −1 | NR | R |
| 26 | TCGA-61-2097 | 0 | NR | R |

TABLE 16
List of patients examined with cumulative scores and response
as predicted for non-responderspatients by the response
predictor as compared to validation dataset.

| | | Cumulative | Predicted | Clinical |
|---|---|---|---|---|
| S. No. | TCGA IDF2:F2:120 | score | Response | response |
| 1 | TCGA-61-1738 | −6 | NR | NR |
| 2 | TCGA-23-1027 | −2 | NR | NR |
| 3 | TCGA-04-1364 | 1 | NR | NR |
| 4 | TCGA-09-0366 | 1 | NR | NR |
| 5 | TCGA-10-0934 | 1 | NR | NR |
| 6 | TCGA-10-0938 | 3 | R | NR |
| 7 | TCGA-13-0723 | 1 | NR | NR |
| 8 | TCGA-13-0724 | −3 | NR | NR |
| 9 | TCGA-13-0755 | 7 | R | NR |
| 10 | TCGA-13-0805 | 3 | R | NR |
| 11 | TCGA-13-1477 | 1 | NR | NR |
| 12 | TCGA-24-0980 | 0 | NR | NR |
| 13 | TCGA-24-1928 | −1 | NR | NR |
| 14 | TCGA-25-1316 | 1 | NR | NR |
| 15 | TCGA-25-1628 | −2 | NR | NR |
| 16 | TCGA-31-1953 | −2 | NR | NR |
| 17 | TCGA-61-1733 | 0 | NR | NR |
| 18 | TCGA-61-1901 | 4 | R | NR |
| 19 | TCGA-61-2000 | 1 | NR | NR |
| 20 | TCGA-61-2110 | 6 | R | NR |

TABLE 17
Number of results as obtained by the response predictor
that were positive, negative, false positives, false
negatives. The table also shows the percentage sensitivity,
specificity and correlation of the tool.

| | |
|---|---|
| n | 46 |
| True Positive | 22 |
| False Positive | 5 |
| True Negative | 15 |
| False Negative | 4 |
| PPV | 85% |
| NPV | 75% |
| Sensitivity | 81% |
| Specificity | 79% |
| Correlation | 80% |

### Example 9

Embodiments herein can contribute to enhanced drug sensitivity or resistance studies by identifying multi-omics signatures using stratification. Embodiments herein can identify mutations or copy number variants or mRNA expressions changes that directly contribute to enhance drug resistance or sensitivity by using the inverse IC₅₀ values. In order to identify sensitive and resistance biomarkers high throughput in vitro screening data of the candidate drug on cancer cell lines and cancer cell line genomics was used. To generate response curves between biomarkers with alterations versus wild type biomarker populations, candidate biomarkers were chosen from drug master network.

During the initial analysis, embodiments herein can identify novel altered biomarkers that were capable of impacting either drug resistance or sensitivity. The biomarkers predicted were validated with validation dataset.

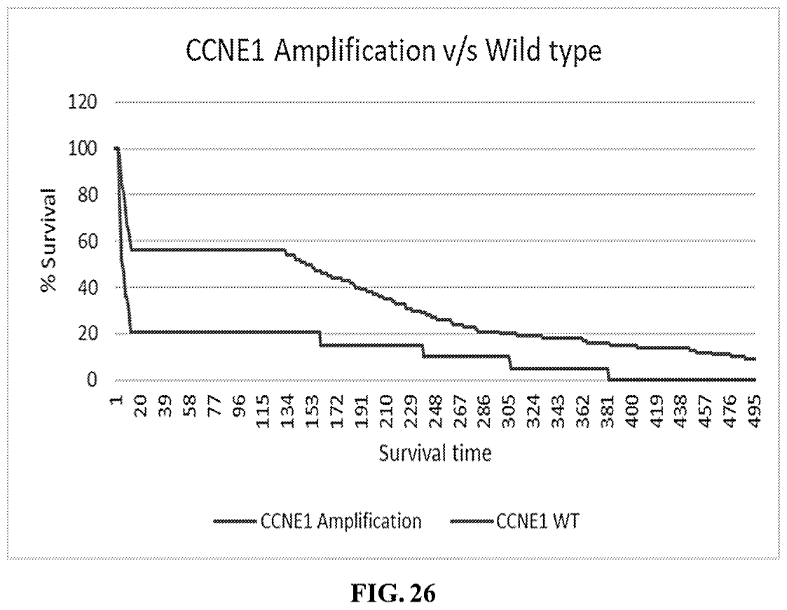

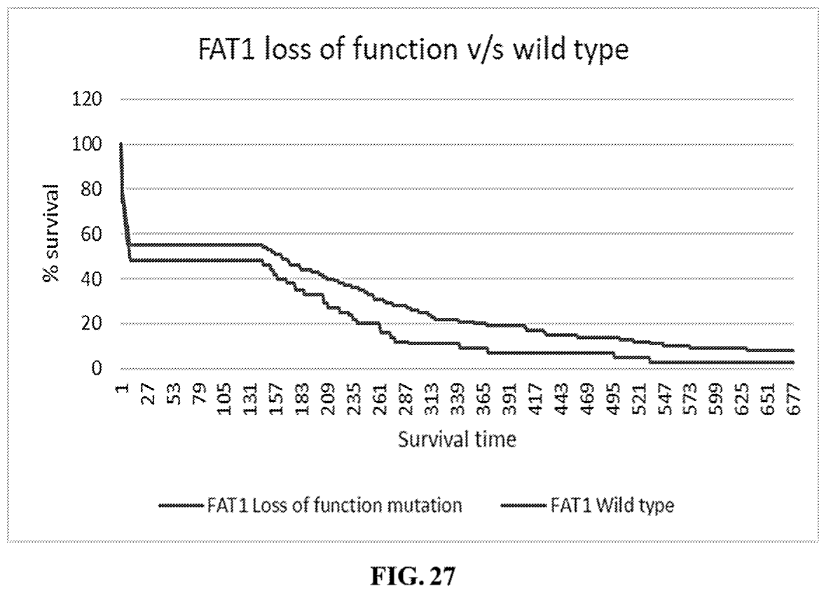

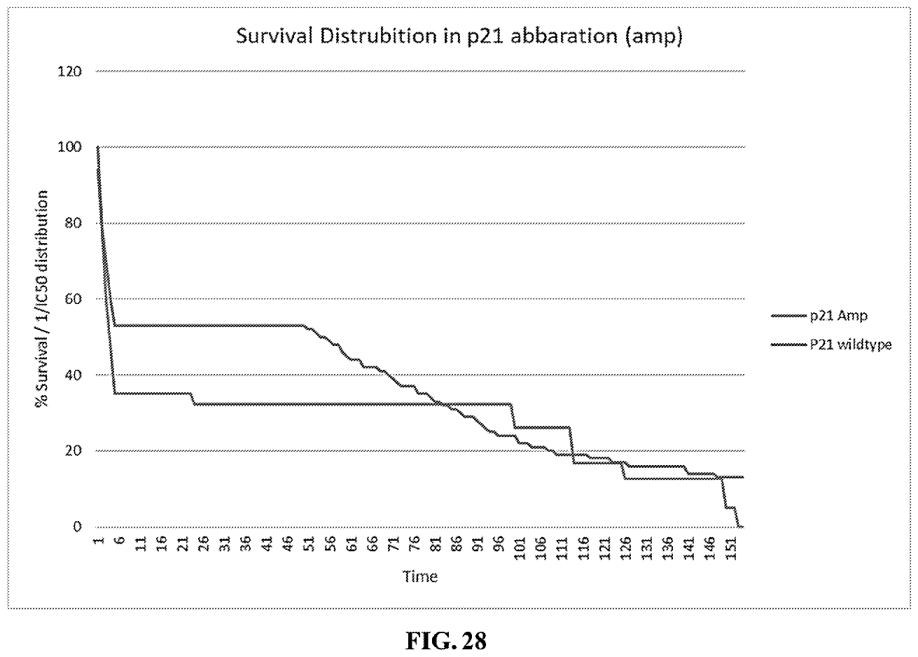

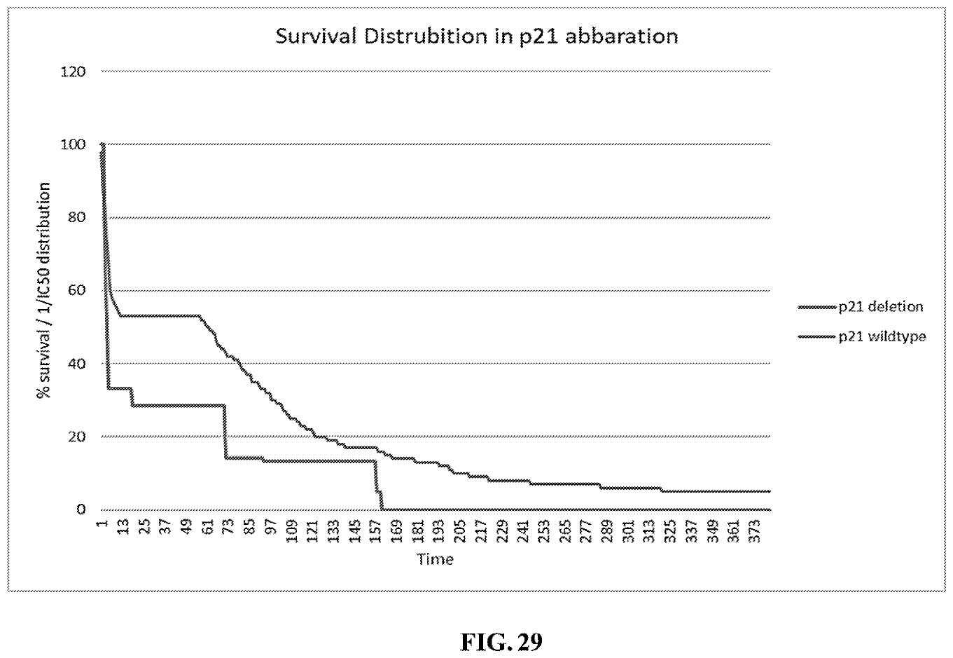

Results/Observations: FAT1 a LOF mutation as a resistance loop for efficacy of the drug Palbociclib was identified as a novel biomarker by embodiments herein. FIG. 27 is a graphical representation of response curves of the cell lines with FAT1 loss of function mutation as compared to wildtype cell lines. Embodiments herein can be used to generate response curves to depict the bi-phasic role of CDKN1A on the efficacy of the drug Palbociclib. FIG. 28 is the system predicted graphical representation of the effect of amplification of CDKN1A on Palbociclib drug efficacy. FIG. 29 is the algorithm predicted graphical representation of the effect of deletion of CDKN1A on Palbociclib drug efficacy. FIG. 35 is a schematic representation of the role of unaltered CDKN1A on drug efficacy as is present in literature evidence. FIG. 26 is a schematic representation of the role of amplified CDKN1A on drug efficacy as is present in literature evidence. FIG. 27 is a schematic representation of the role of deleted CDKN1A on drug efficacy as is present in literature evidence.

### Example 10: System 100

The response prediction model of cancer cell lines with AML to standard induction chemotherapy drugs Cytarabine and Anthracycline was done using the integrated approach. The dataset used for training the AI Model in order to validate the models is as follows: Dataset—Beat AML; Drug—7+3 standard induction chemotherapy; Source: cBioPortal.

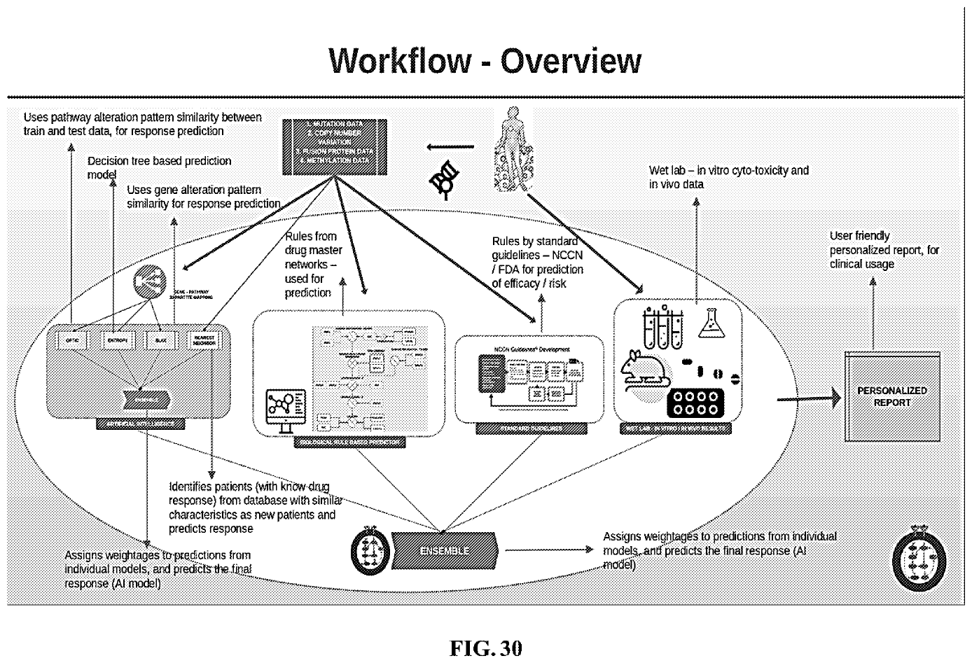

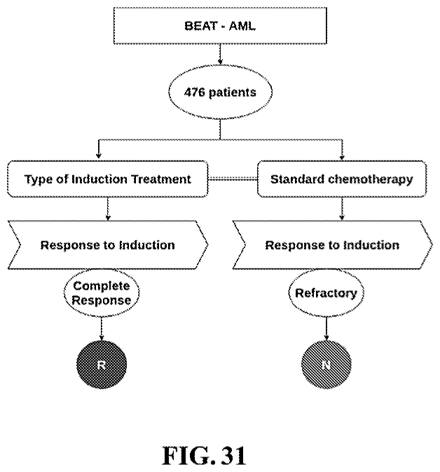

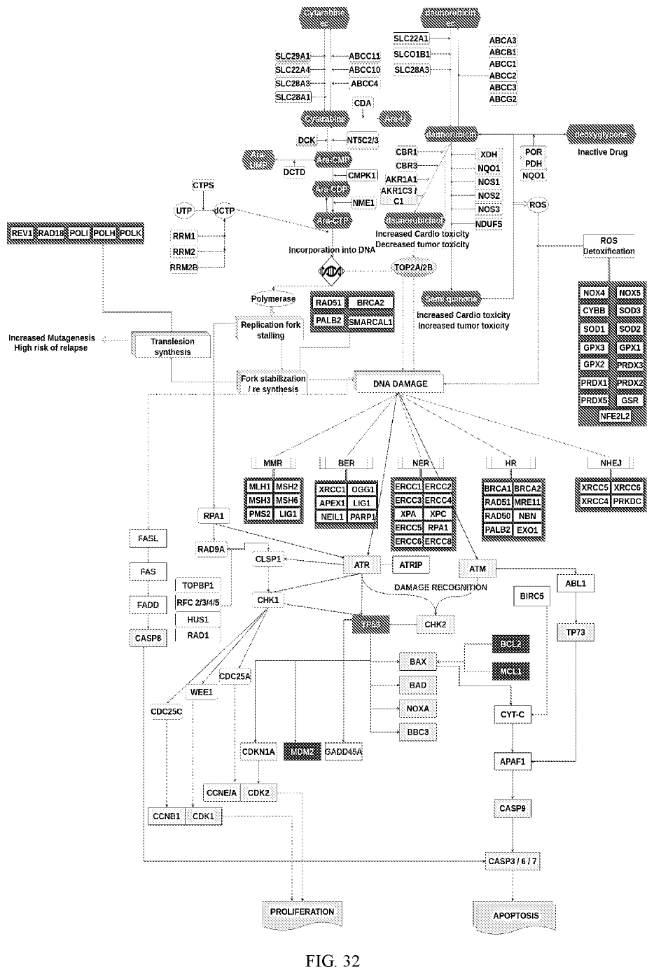

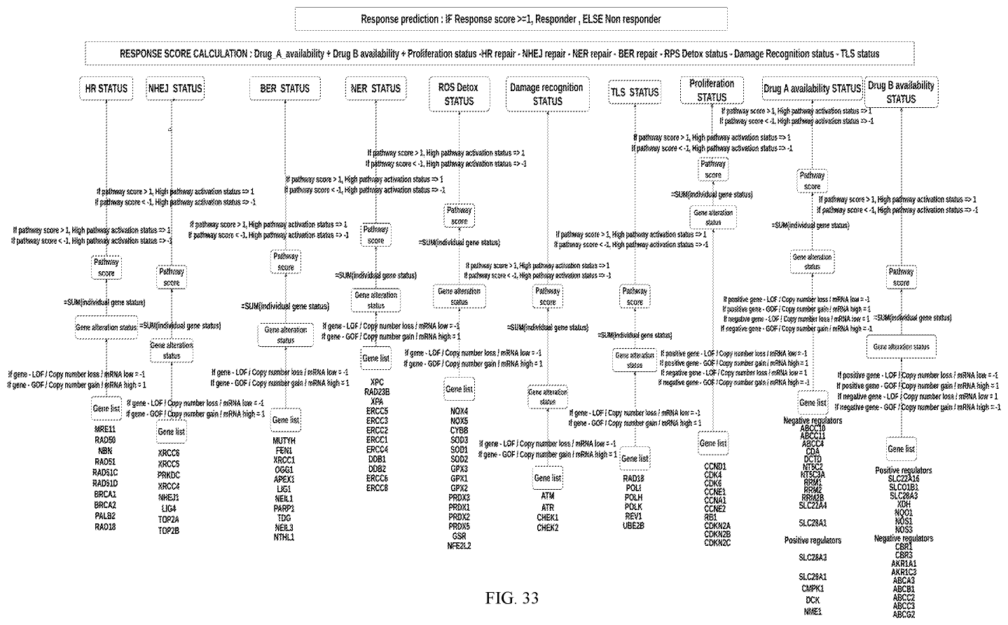

Results/Observations: FIG. 30 shows a flowchart representation of the Integrated Approach. Table 18 shows the percentage inhibition for each cell line. A flowchart depicting a patient specific response prediction for the BEAT-AML program, by a system according to embodiments herein, is described in FIG. 31. An example flowchart representation of response prediction based on biological rules in 7+3 standard induction chemotherapy drug system, by a system according to embodiments herein, is described in FIG. 32. FIG. 33 depicts a representation of assigning response scores for response prediction based on biological rules in 7+3 standard induction chemotherapy drug system by a system according to embodiments herein.

Table 19 shows the score of the response as predicted by the individual models of first module 101 and the final cumulative score in order to assess the responsiveness or non-responsiveness of a patient to the particular drug or drug combination. Table 20 shows the response prediction of standard induction chemotherapy in AML patients using computational biology methods.

TABLE 18
Percentage inhibition shown by each cell line to the drugs.

| | | % inhibition @ 25 uM |
|---|---|---|
| | | cytarabine + 5 uM |
| S. No. | Cell line | Daunorubicin |
| 1 | HL60 | 80% |
| 2 | K562 | 20% |
| 3 | U937 | 30% |

TABLE 19
Score of the response as predicted by the individual models of first module
101 and the final cumulative score in order to assess the responsiveness or
non-responsiveness of a patient to the particular drug or drug combination.

| | | | | | | | Wet lab | | Final |
|---|---|---|---|---|---|---|---|---|---|
| | | | | | Rule | Risk | (% | | Clinical |
| Cell | Module 1 | Module 2 | Module 3 | Module 4 | Based | Stratification | inhibition) | Final | response |
| Line | 102 | 104 | 103 | 105 | 108 | 107 | 109 | score | prediction |
| HL60 | −0.18 | −0.22 | 0 | 0 | 0.16 | −0.1 | 80 | 0.17 | R |
| K562 | −0.24 | −0.31 | 0 | 0 | 0.02 | −0.1 | 20 | −0.11 | N |
| U937 | −0.26 | −0.29 | 0 | 0 | −0.07 | −0.1 | 30 | −0.08 | N |

TABLE 20
Response prediction of standard induction chemotherapy in AML patients using computational biology methods.

| | | | | | | | Rule | | |
|---|---|---|---|---|---|---|---|---|---|
| | Actual | | | | | Risk | based | | Predicted |
| | Clinical | Module 1 | Module 2 | Module 4 | Module 3 | score | score | Final | Clinical |
| Patient IDA15:J22 | Response | 102 | 104 | 105 | 103 | 107 | 108 | Score | Response |
| aml_ohsu_2018_16-00264 | N | −0.99 | −0.34 | −0.99 | 0 | 0.01 | 0.02 | −0.128 | N |
| aml_ohsu_2018_16-00525 | N | 0 | −0.24 | −0.33 | 0 | 0.01 | 0 | −0.029 | N |
| aml_ohsu_2018_13-00540 | R | 0.05 | 0.28 | 0.5 | 0 | 0.01 | 0 | 0.044 | R |
| aml_ohsu_2018_13-00572 | R | 0.43 | −0.31 | 0.11 | 0 | −0.1 | 0.057 | 0.02 | R |
| aml_ohsu_2018_15-00482 | R | 0.02 | 0.29 | 0.4 | 0 | 0.1 | 0 | 0.04 | R |

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A method for predicting response of a subject to a drug or a combination of drugs, the method comprising:
predicting, by a processor (106), the response of the subject based on at least one of a first factor, a second factor, a third factor, and a fourth factor, weights assigned of each of the first factor, the second factor, the third factor, and the fourth factor, influencing an outcome of the prediction,
wherein the first factor comprising at least one pathway feature, derived based on multi-omics of the subject is determined, by a first model (102), wherein the at least one pathway feature is obtained by conversion of multi-omics data pertaining to the subject through in-house bi-partite mapping of genes of the subject into cancer pathways;
wherein the second factor comprising a plurality of decision trees is determined, by a second model (103), wherein the plurality of decision trees, derived based on a random forest technique, correspond to the at least one genetic feature derived based on the multi-omics of the subject,
wherein the third factor comprising a plurality of decision trees is determined, by a third model (104), wherein the plurality of decision trees, derived based on a random forest technique, correspond to the at least one pathway feature derived based on multi-omics of the subject; and
wherein the fourth factor defining a similarity, determined using a nearest neighbour technique, between the at least one pathway feature derived based on multi-omics of the subject and the at least one pathway feature derived based on genomics of at least one reference subject is determined, by a fourth model (105).

2. The method, as claimed in claim 1, wherein the first model (102) is built by identifying correlating the multi-omics features of the subject, comprising at least one of gene mutation information, CNV, methylation, fusion, and mRNA, with response of the at least one reference subject to at least one of a reference drug and a reference combination of drugs, or
wherein the second model (103), the third model (104), and the fourth model (105) are trained using the at least one pathway feature derived based on the multi-omics of the at least one reference subject, and the response of the at least one reference subject to at least one of the reference drug and the reference combination of drugs, or
wherein the weights are assigned to each of the first factor, the second factor, the third factor, and the fourth factor based on at least one of a relevance and accuracy of at least one of the first model (102), the second model (103), the third model (104), and the fourth model (105).

3. (canceled)

4. (canceled)

5. The method, as claimed in claim 1, wherein the method further comprises predicting, by a MDS module (111), a functionality of a mutation, as one of a Gain of Function (GOF), Loss of Function (LOF), Conservation of Function (COF), and Switch of function (SOF), and
wherein the prediction is performed using gene models comprising at least one of a wild type protein model and a mutant protein model, wherein the gene models are created using pattern recognition, and
wherein a PDB protein structure is used for constructing the wild type protein model and the mutant protein model, and an activity projection for the wild type protein model and the mutant protein model.

6. (canceled)

7. (canceled)

8. The method as claimed in claim 1, further comprising performing, by a second module (107), subject risk stratification, by classifying at least one of a plurality of subjects into a plurality of sub-groups based on predefined rules, wherein the plurality of subjects are stratified as one of favorable risk, intermediate risk, and adverse risk, based on genomic characteristics of each of the subjects.

9. The method as claimed in claim 8, wherein the method of performing risk stratification comprises:
identifying, by the second module (107), at least one statistically significant genomic characteristic in the subject; based on at least one of pre-defined guidelines derived from datasets on genomics alterations showing favorable therapeutic response and scope of survival;
assessing, by the second module (107), a risk level of the subject by classifying the subject into at least one of a plurality of sub-groups selected from at least one category comprising a favorable risk, an intermediate risk, and an adverse risk, based on the identified genomic characteristics; and
predicting, by the second module (107), the response based on the assessed risk level.

10. The method as claimed in claim 1, further comprising
determining, by a third module (108), at least one multi-omics alteration in the subject based on at least one predefined biological rule affecting at least one of drug sensitivity, and drug resistance, in the subject, wherein the at least one biological rule is derived from at least one deregulation in functional status of at least one of a pathological target, a pathway effector and a pathway regulator; and
predicting the response based on the multi-omics alteration in the subject.

11. A system (100) for predicting response of a subject to a drug or a combination of drugs, the system (100) comprising:
a prediction engine (112), wherein the prediction engine (112) combines response of the subject predicted by at least one of a first module (101), a second module (107), and a third module (108) based on priorities corresponding to the predictions performed by any or a combination of the first module (101), the second module (107), and the third module (108)
wherein the first module (101) comprises a plurality of models configured to predict the response of the subject based on a plurality of factors comprising multi-omics of the subject, at least one genetic feature derived based on the multi-omics of the subject, at least one pathway feature derived based on multi-omics of the subject, and at least one pathway feature derived based on genomics of at least one reference subject;
wherein the second module (107) is configured to predict the response of the subject based on a risk level of the subject to an oncological condition, wherein the risk level is assessed based on genomic characteristics of the subject;
wherein the third module (108) is configured to predict the response of the subject based on multi-omics alteration in the subject, wherein the multi-omics alteration is determined based on at least one predefined biological rule affecting at least one of a drug sensitivity and a drug resistance, in the subject.

12. The system (100), as claimed in claim 11, wherein the plurality of models comprises a first model (102) configured to determine a first factor comprising at least one pathway feature, derived based on the multi-omics of the subject, wherein the at least one pathway feature is obtained by conversion of multi-omics data pertaining to the subject through in-house bi-partite mapping of genes of the subject into cancer pathways, or
wherein the plurality of models comprises a second model (103) configured to determine a second factor comprising a plurality of decision trees, wherein the plurality of decision trees, derived based on a random forest technique, correspond to the at least one genetic feature derived based on the multi-omics of the subject, or
wherein the plurality of models comprises a third model (104) configured to determine a third factor comprising a plurality of decision trees wherein the plurality of decision trees, derived based on a random forest technique, correspond to the at least one pathway feature derived based on multi-omics of the subject, or
wherein the plurality of models comprises a fourth model (105) configured to determine a fourth factor defining a similarity, determined using a nearest neighbour technique, between the at least one pathway feature derived based on the multi-omics of the subject and the at least one pathway feature derived based on genomics of at least one reference subject.

13. (canceled)

14. (canceled)

15. (canceled)

16. The system (100), as claimed in claim 11, wherein the first module (101) predicts the response of the subject based on weights assigned of each of the plurality of factors, wherein the weights are at least one of determined and updated, by at least one Machine Learning (ML) model, based on at least one of a relevance and accuracy, of at least one of the first model (102), the second model (103), the third model (104), and the fourth model (105), and
wherein the first model (102) is built by identifying correlation of the multi-omics features of the subject, comprising at least one of gene mutation information, CNV, methylation, fusion, and mRNA, with response of the at least one reference subject to at least one of a reference drug and a reference combination of drugs.

7. (canceled)

18. The system (100), as claimed in claim 12, wherein the second model (103), the third model (104), and the fourth model (105) are trained using the at least one pathway feature derived based on the multi-omics of the at least one reference subject, and the response of the at least one reference subject to at least one of the reference drug and the reference combination of drugs.

19. (canceled)

20. (canceled)

21. The system (100), as claimed in claim 11, wherein the system (100) further comprises a MDS module (111), wherein the MDS module (111) is configured to predict a functionality of mutation as one of a Gain of Function (GOF), Loss of Function (LOF), Conservation of Function (COF), and Switch of function (SOF).

22. (canceled)

23. A method for identification and selection of biomarkers, cell lines, target patient and target indication for a drug candidate in drug discovery and development, said method, by a system (200), comprising
identifying, by biomarker selection module (201), at least one biomarker, capable of affecting at least one of sensitivity and resistance of a subject to the drug candidate, as a positive biomarker or a negative biomarker, based on an effect of at least one biomarker on a pharmacological target, wherein the identification is performed by constructing a Drug master network for the pharmacological target using pathway features comprising at least one of regulators of a target pathway and effectors of the target pathway, wherein the pathway features comprise at least one of upstream regulators, upstream effectors, downstream effectors, downstream regulator, parallel pathway regulators and pathway cross talk effectors; and
identifying, by a cell line selection module (202), at least one cell line, or subset thereof, comprising alterations in at least one of the identified at least one biomarker and pharmacokinetic determinants of drug resistance, wherein the pharmacokinetic determinants comprise factors affecting intracellular drug transport and drug metabolism.

24. The method as claimed in claim 23, wherein the method further comprising
identifying, by the biomarker selection module (201), at least one of a drug sensitive pathway loop and a drug resistant pathway loop, comprising the identified at least one biomarker in the drug master network,
OR determining, by a patient selection module (203), statistically significant multi-omics alterations of drug pathway, by variant calling, in each of the at least one identified cell line by integrating multi-omics data of the at least one identified cell line and the drug master network; and
selecting, by the patient selection module (203), the target patient having favourable genomics by assigning weightages to pathway players comprising the identified at least one biomarker, based on a frequency of the statistically significant multi-omics alterations, and the pharmacokinetic determinants,
OR determining, by an indication selection module (204), statistically significant multi-omics alterations for a drug candidate, by variant calling, in each of the at least one identified cell line by integrating patient multi-omics data of the at least one identified cell line to the drug master network; and
selecting, by the indication selection module (204), the target indication having favourable multiomics for a drug candidate by assigning weightages to pathway players comprising identified biomarkers, based on a frequency of the statistically significant multi-omics alterations, and the pharmacokinetic determinants.

25. (canceled)

26. (canceled)

27. The method as claimed in claim 23, the method further comprising: determining, by a drug transporter identification module (205), genomic factors affecting the pharmacokinetic determinants of drug resistance for the drug candidate using IC50 values and multi-omics alterations of sensitive cell lines; and identifying, by the drug transporter identification module (205), a statistically significant drug transporter by determining genomic factors affecting the pharmacokinetic determinants of drug resistance for the drug candidate using the IC50 values and the multi-omics alterations of sensitive cell lines.

28. A system (200) for identification and selection of biomarkers, cell lines, target patient and target indication for a drug candidate in drug discovery and development, the system (200), comprising
a biomarker selection module (201), and
a cell line selection module (202);
wherein the biomarker selection module (201) is configured to identify at least one biomarker, capable of affecting at least one of sensitivity and resistance of a subject to the drug candidate, as a positive biomarker or a negative biomarker, based on an effect of the at least one biomarker on a pharmacological target, wherein the identification is performed by constructing a Drug master network for the pharmacological target using pathway features comprising at least one of regulators of a target pathway and effectors of the target pathway, wherein the pathway features comprise at least one of upstream regulators, upstream effectors; downstream effectors, downstream regulator, parallel pathway regulators and pathway cross talk effectors; and
wherein the cell line selection module (202) is configured to identify at least one cell line, or subset thereof, comprising alterations in at least one of the identified at least one biomarker and pharmacokinetic determinants of drug resistance, wherein the pharmacokinetic determinants comprise factors affecting intracellular drug transport and drug metabolism.

29. The system (200), as claimed in claim 28, wherein the biomarker selection module (201) is further configured to identify at least one of drug sensitive pathway loop and drug resistant pathway loop, comprising the identified at least one biomarker in the drug master network.

30. The system (200), as claimed in claim 28, wherein the system (200) further comprises a patient selection module (203), wherein the patient selection module (203) is configured to:
determine statistically significant multi-omics alterations of drug pathway, by variant calling; in each of the at least one identified cell line by integrating multi-omics data of the at least one identified cell line and the drug master network; and
select the target patient having favourable genomics by assigning weightages to pathway players comprising the identified at least one biomarker, based on a frequency of the statistically significant multi-omics alterations, and the pharmacokinetic determinants.

31. The system (200), as claimed in claim 28, wherein the system (200) further comprises an indication selection module (204), wherein the indication selection module (204) is configured to: determine statistically significant multi-omics alterations for a drug candidate, by variant calling, in each of the at least one identified cell line by integrating patient multi-omics data of the at least one identified cell line to the drug master network; and select the target indication having favourable multi-omits for a drug candidate by assigning weightages to pathway players comprising identified biomarkers, based on a frequency of the statistically significant multi-omics alterations, and the pharmacokinetic determinants.

32. The system (200), as claimed in claim 28, wherein the system (200) further comprises a drug transporter identification module (205), wherein the drug transporter identification module (205) is configured to:
determine genomic factors affecting the pharmacokinetic determinants of drug resistance for the drug candidate using IC50 values and multi-omics alterations of sensitive cell lines; and
identify a statistically significant drug transporter by determining genomic factors affecting the pharmacokinetic determinants of drug resistance for the drug candidate using the IC50 values and the multi-omics alterations of sensitive cell lines.
